(19) United States
(12) Patent Application Publication
(10) Pub. No.: US 2022/0370814 A1
(43) Pub. Date: Nov. 24, 2022

(54) ESTHETIC APPARATUS USEFUL FOR INCREASING SKIN REJUVENATION AND METHODS THEREOF

(71) Applicant: VENUS CONCEPT LTD., Yokoneam Illit (IL)

(72) Inventors: Haim Epshtein, Pardes Hana-Karkur (IL); Vadim Polyakov, Petach Tikva (IL); Oded Ron Edoute, Tel Aviv (IL); Orit Ron Edoute, Tel Aviv (IL); Itzhak Kremin, Givatayim (IL)

(73) Assignee: VENUS CONCEPT LTD., Yokeneam Illit (IL)

(21) Appl. No.: 17/859,933

(22) Filed: Jul. 7, 2022

Related U.S. Application Data
(63) Continuation-in-part of application No. 16/864,862, filed on May 1, 2020.
Application No. 16/864,862 is a continuation of application No. 15/728,560, filed on Oct. 10, 2017, now Pat. No. 10,661,093.
Application No. 15/728,560 is a continuation of application No. 15/341,010, filed on Nov. 2, 2016, now Pat. No. 9,901,743.
Application No. 15/341,010 is a continuation of application No. 14/845,315, filed on Sep. 4, 2015, now Pat. No. 9,814,897.
Application No. 14/845,315 is a division of application No. 14/489,572, filed on Sep. 18, 2014, now Pat. No. 9,694,194.
Application No. 14/489,572 is a division of application No. 13/954,320, filed on Jul. 30, 2013, now Pat. No. 8,979,727.
Application No. 13/954,320 is a division of application No. 13/001,834, filed on Feb. 1, 2011, now Pat. No. 8,998,791.
(60) Provisional application No. 63/219,385, filed on Jul. 8, 2021.
(60) Provisional application No. 61/112,783, filed on Nov. 10, 2008.
(60) Provisional application No. 61/076,652, filed on Jun. 29, 2008.

(51) Int. Cl. A61N 2/00; A61N 1/32; A61N 1/40; A61N 2/02; A61N 7/02; A61F 7/00; A61N 5/00; A61N 5/06

(52) U.S. Cl. CPC A61N 2/002; A61N 1/328; A61N 1/403; A61N 2/02; A61N 7/02; A61F 7/00; A61N 2/004; A61F 7/007; A61N 5/00; A61N 5/0625; A61B 18/203

(57) ABSTRACT

An integrated system for increasing skin rejuvenation of a region of a patient's skin and treating subcutaneous fat associated with the region is provided. The system includes at least one first applicator having one or more RF electrodes adapted to be placed on the region of the patient's skin without penetrating the skin. At least one of said RF electrodes is configured to apply RF energy to the region of the patient's skin to heat up to a temperature in a specified temperature range. The system also includes at least one second applicator, in communication with an optical system. The optical system includes at least one laser source and is adapted to provide homogeneous illumination to the skin surface to effect at least one target tissue at a given depth range and power density range.

### CROSS-REFERENCE TO RELATED APPLICATIONS

This application claims priority to U.S. patent application Ser. No. 63/219,385 filed Jul. 8, 2021, and is also a Continuation-in-Part of U.S. patent application Ser. No. 16/864,862 filed May 1, 2020. U.S. patent application Ser. No. 16/864,862 is a Continuation of U.S. patent application Ser. No. 15/728,560 filed Oct. 10, 2017, now U.S. Pat. No. 10,661,093, issued May 26, 2020, which is a Continuation of U.S. patent application Ser. No. 15/341,010 filed Nov. 2, 2016, now U.S. Pat. No. 9,901,743, issued Feb. 27, 2018, which is a Continuation of U.S. patent application Ser. No. 14/845,315, filed Sep. 4, 2015, now U.S. Pat. No. 9,814,897, issued Nov. 14, 2017, which is a Divisional of U.S. patent application Ser. No. 14/489,572, filed Sep. 18, 2014, now U.S. Pat. No. 9,694,194 issued on Jul. 4, 2017, which is a Divisional of U.S. patent application Ser. No. 13/954,320, filed Jul. 30, 2013, now U.S. Pat. No. 8,979,727, issued on Mar. 17, 2015, which is a Divisional of U.S. patent application Ser. No. 13/001,834, filed Feb. 1, 2011, now U.S. Pat. No. 8,998,791, issued on Apr. 7, 2015, which is a National Phase of International Application number PCT/IL2009/000644, filed Jun. 29, 2009, which claims priority from U.S. Provisional Patent Application No. 61/112,783, filed on Nov. 10, 2008, and U.S. Provisional Patent Application No. 61/076,652, filed on Jun. 29, 2008. The entire contents of each of the foregoing are incorporated herein by reference.

### FIELD OF THE INVENTION

This invention generally relates to a device used to improve skin viability and skin rejuvenation, and a method of using the device.

### BACKGROUND OF THE INVENTION

Improving the appearance of the skin has been the goal of many esthetic products and procedures for many years, since a tight skin, without wrinkles or cellulite, has a younger and more appealing appearance. Apart from age related changes, the skin also suffers from exposure to chemical and physical injuries, such as tobacco, cosmetics, esthetics and radiation from the sun and other sources. Those factors contribute to the decrease in collagen production, to reduced elasticity, and the appearance of wrinkles.

A few main approaches to tightening of the skin are common practice today. The surgical approach carries disadvantages related to the anesthesia, the surgical complications, and the healing process, which may cause scars. The chemical peel approach usually involves injury to the outermost layer of the skin—the epidermis—which may cause discoloration. Since collagen fibers are found in the dermis—the subcutaneous layer of the skin, and since heat was shown to contract these fibers and stimulate their production [Zelickson B D, Kist D, Bernstein E, Brown D B, Ksenzenko S, Burns J, Kilmer S, Mehregan D, Pope K. Histological and ultrastructural evaluation of the effects of a radiofrequency-based nonablative dermal remodeling device: a pilot study. Arch Dermatol. 2004 February; 140(2):204-9], methods of differentially heating the dermis (deep tissue diathermy) have recently arisen.

A unique method of treating the dermis is called Pulsed Electromagnetic Fields (PEMF) therapy. This method usually employs electromagnetic radiation of different frequencies—ranging from static magnetic fields, through extremely low frequencies (ELF) to higher radiofrequencies (RF)—administered in pulses.

PEMF works in few ways. Due to the radiation absorbed in the tissue, it can be heated to various temperatures, depending on the power applied, the frequency transmitted, and more importantly the tissue characteristics. Eventually, the tissue can be warmed to denaturation temperatures, which cause coagulation necrosis and tissue damage. It can also be heated to lower temperatures, which proved to result in the afore-mentioned contraction of collagen fibers.

Another modus operandi involves non thermal effects. These rely on specific tissue components and their reaction to the applied radiation characteristics. These effects might be due to large charged molecules and their reaction to various frequencies and frequency harmonies, charged small ions in the cell membranes affecting the cells function and reactions to hormones and chemical signals, charged small ions in the extracellular space and likely also via other less understood mechanisms.

Furthermore, applying the radiation in pulses was also found to have non thermal effects. Yet more, only a specific combination of frequency, duty cycle and transmitted power intensity achieve a specific tissue response. Recent scientific research has confronted these challenges and found the PEMF characteristics needed for the desired biophysical response.

It is now commonly accepted that weak electromagnetic fields (EMF) administered in pulses are capable of initiating various healing processes in fractures, multiple sclerosis and Parkinson's disease, and even delivering pain relief; however it seems that most of the conditions that seem most likely to respond to PEMF are musculoskeletal. The FDA has allowed the use of pulsed radiofrequency electromagnetic field for treatment of pain and edema in superficial soft tissues two decades ago. [Rosch, P. J., Markov, M. S., eds. Bioelectromagnetic Medicine, Marcel Dekker, NY, 251-264].

The use of PEMF can also be recruited for cosmetic purposes as described above. Several studies have addressed the effect of PEMF on dermal components. For example, in vivo trials showed that pulsed electromagnetic field of certain field intensities and frequencies increased dermal collagen synthesis [Ahmadian S, Zarchi S R, Bolouri B. Effects of extremely-low-frequency pulsed electromagnetic fields on collagen synthesis in rat skin. Biotechnol Appl Biochem. 2006 February; 43(Pt 2):71-5]. This new formed collagen increases skin elasticity and rejuvenates its appearance.

In vitro trials showed that PEMF increased the degree of endothelial cell tubulization and proliferation, and augmented angiogenesis primarily by stimulating endothelial release of FGF-2, inducing paracrine and autocrine changes in the surrounding tissue [Tepper O M et al. Electromagnetic fields increase in vitro and in vivo angiogenesis through endothelial release of FGF-2. FASEB J. 2004 August; 18(11):1231-3. Epub 2004 Jun. 18]. Angiogenesis, the sprouting of new blood vessels, increases blood flow to the tissue, which in turn increases oxygen and nutritional substances delivery to the tissue. This enhanced anabolic effect is most beneficial for an injured tissue, promoting rapid and improved healing. The growth factor released further enhances the healing process, both in quality and time of improvement.

The effect of PEMF on tissues was utilized in various applications. For example, US20050182462A1 discloses healthy deep tissue heating using PEMF for the purpose of causing contraction and tightening of the skin.

PEMF has also been used to improve skin wound healing. For example, WO08064272 discloses a method of treating a severe diabetic ulcer using PEMF. The patent also discloses the addition of intermittent compression therapy (ICT) and the use of low intensity ultrasound (up to 50 W/cm²), the latter aimed at inhibiting microbial growth.

Other methods of heating the dermis used non pulsating RF radiation, applied by antenna or electrodes. For example, WO98005380 discloses a method of tightening skin using an RF electromagnetic energy delivery device.

Improving the results of skin tightening based on dermis diathermy is still a long felt need.

### SUMMARY OF THE INVENTION

It is one object of the present invention to provide a system (10) adapted to increase skin rejuvenation of a region of a patient's skin, said system comprising
a. a pulsed electromagnetic field (PEMF) frequency generator (2) for constantly providing electromagnetic pulses to said region of a patient's skin; and,
b. a deep tissue diathermy device (4) for constantly applying heat to said region of a patient's skin up to temperature T;

said system (10) is adapted for simultaneously applying heat and PEMF to said region of a patient's skin; wherein application of said system increases said skin rejuvenation such that said skin rejuvenation increase (SRI) is greater than the sum of said SRI provided by electromagnetic pulses increase and said SRI provided by said deep tissue diathermy device increase.

It is another object of the present invention to provide the system as defined above, wherein said electromagnetic pulse is a triangular shaped at frequency of 25 Hz and intensity of 20 Gauss.

It is another object of the present invention to provide the system as defined above, wherein said electromagnetic pulse is square shaped at a frequency of 16 Hz, duration of about 5 milliseconds and intensity of 12 Gauss.

It is another object of the present invention to provide the system as defined above, wherein said deep tissue diathermy device (4) is selected from any device emitting RF radiation or any means adapted for producing electrical current absorbed by subcutaneous tissue (e.g., subcutaneous fat).

It is another object of the present invention to provide the system as defined above, wherein said deep tissue diathermy device (4) additionally comprises:
a. at least one electrical output device adapted to generate RF electromagnetic energy;
b. at least two electrodes electrically coupled to said electrical output device and placed on said skin region, wherein all said electrodes are adapted to simultaneously apply said RF energy to said skin region.

It is another object of the present invention to provide the system as defined above, wherein said deep tissue diathermy device (4) additionally comprises:
a. at least one electrical output device adapted to generate electrical current;
b. at least two electrodes electrically coupled to said electrical output device and placed on said skin region, wherein all said electrodes are adapted to simultaneously apply said electrical current to said skin region.

It is another object of the present invention to provide the system as defined above, wherein said system reduces side effects and/or harmful effects of said electromagnetic pulses and/or said deep tissue diathermy such that said reduction of said side effects and/or said harmful effects is greater than the sum of said reduction of said electromagnetic pulses and/or said reduction of said deep tissue diathermy.

It is another object of the present invention to provide the system as defined above, wherein said system additionally comprising a control system (6) adapted to regulate said electromagnetic pulses and/or said electromagnetic pulses.

It is another object of the present invention to provide the system as defined above, wherein said pulsed electromagnetic frequency generator is adapted to provide a dynamic magnetic field such that said pulses vary with time.

It is another object of the present invention to provide the system as defined above, wherein the shape of said electromagnetic pulse is selected in a non-limiting manner from a group consisting of square wave, a sine wave, a triangular wave, sawtooth wave, ramp waves, spiked wave or any other mathematical combination.

It is another object of the present invention to provide the system as defined above, wherein the magnetic field intensity B of each pulse applied by said pulsed electromagnetic frequency generator (2) ranges between about 0 and about 3 Tesla.

It is another object of the present invention to provide the system as defined above, wherein the duration of each pulse applied by said pulsed electromagnetic frequency generator (2) ranges between about 3 and about 1000 milliseconds.

It is another object of the present invention to provide the system as defined above, wherein the frequency F applied by the pulses of said pulsed electromagnetic frequency generator (2) ranges between about 1 Hz and about 1M Hz.

It is another object of the present invention to provide the system as defined above, wherein the power applied by the RF electrode ranges between about 1 and about 150 watts per pulse.

It is another object of the present invention to provide the system as defined above, wherein said deep tissue diathermy device (4) is selected in a non-limiting manner from a group consisting of an ultrasonic diathermy, an optical device, electromagnetic induction, sound waves emitting instrument, direct heat applying instrument, or from any other means of heating subcutaneous tissue to temperature T.

It is another object of the present invention to provide the system as defined above, wherein said optical device is adapted to emit light in wavelengths absorbed by subcutaneous tissue such that said subcutaneous tissue is heated.

It is another object of the present invention to provide the system as defined above, wherein said sound waves emitting instrument is adapted to emit sound waves absorbed by the subcutaneous tissue such that said subcutaneous tissue is heated.

It is another object of the present invention to provide the system as defined above, wherein said temperature T is higher than about 30 and lower than about 80 degrees.

It is another object of the present invention to provide the system as defined above, wherein said power supply and control system (6) monitors physical tissue parameters and changes applied heat and electromagnetic pulses accordingly.

It is another object of the present invention to provide the system as defined above, wherein said power supply and control system (6) additionally comprising:
a. processing means, adapted to store in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing means; adapted to sense electromagnetic radiation and heat radiation parameters selected from a group consisting of time t of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. regulating means, adapted to allow said electromagnetic radiation and heat radiation if said parameters are within said safe treatment parameters and to stop the electromagnetic radiation if the radiation parameters are in said unsafe treatment parameters.

It is another object of the present invention to provide the system as defined above, wherein said power supply and control system (6) includes a mechanism for skin cooling.

It is another object of the present invention to provide the system as defined above, wherein said system (10) is encased in at least one platform.

It is another object of the present invention to provide the system as defined above, wherein said pulsed electromagnetic frequency generator (2) and said deep tissue diathermy device (4) have more than one applicator to treat more than one body part simultaneously.

It is another object of the present invention to provide the system as defined above, wherein said pulsed electromagnetic frequency generator (2) has electrostatic shielding.

It is another object of the present invention to provide the system as defined above, especially adapted to increase skin rejuvenation in the immediate (short) range.

It is another object of the present invention to provide the system as defined above, especially adapted to increase skin rejuvenation in the intermediate range.

It is another object of the present invention to provide the system as defined above, especially adapted to increase skin rejuvenation in the long range.

It is another object of the present invention to provide the system as defined above, wherein said system is especially adapted to operate according to IEC selected from a group consisting of IEC 60601-2-35, IEC 60601-2-33, IEC 60601-2-29, IEC 60601-2-9, IEC 60601-2-5, IEC 60601-2-3, IEC 60601-1-8, IEC 60601-1-6, IEC 60601-1-4, IEC 60601-1-3, IEC 60601-1-2, IEC 60601-1-1 or any combination thereof.

It is another object of the present invention to provide a method (400) of increasing skin rejuvenation of a region of a patient's skin. The method comprises steps selected inter alia from:
a. obtaining (i) pulsed electromagnetic frequency generator; and, (ii) a deep tissue diathermy device;
b. applying heat to a subcutaneous tissue within said region up to temperature T; and,
c. applying pulses of electromagnetic field to said region;

wherein said increasing of said skin rejuvenation is greater than the sum of said applying heat to a subcutaneous tissue within said region increase and said applying pulses electromagnetic therapy to said region increase.

It is another object of the present invention to provide a method (410) of increasing skin rejuvenation of a region of a patient's skin. The method comprises steps selected inter alia from:
a. obtaining (i) pulsed electromagnetic frequency generator; and, (ii) a deep tissue diathermy device;
b. applying pulses of electromagnetic field to said region; and,
c. applying heat to a subcutaneous tissue within said region up to temperature T;

wherein said increasing of said skin rejuvenation is greater than the sum of said applying heat to a subcutaneous tissue within said region and said applying pulses electromagnetic therapy to said region

It is another object of the present invention to provide a method (420) of increasing skin rejuvenation of a region of a patient's skin. The method comprises steps selected inter alia from:
a. obtaining (i) pulsed electromagnetic frequency generator; and, (ii) a deep tissue diathermy device;
b. applying heat to a subcutaneous tissue within said region up to temperature T; whilst simultaneously applying pulses of electromagnetic field to said region;

wherein said increasing of said skin rejuvenation is greater than the sum of said applying heat to a subcutaneous tissue within said region and said applying pulses electromagnetic therapy to said region.

It is another object of the present invention to provide the methods as defined above, wherein said system reduces [symbol] and/or [symbol] of said electromagnetic pulses and/or said deep tissue diathermy such that said reduction of said side effects and/or said harmful effects is greater than the sum of said reduction of said electromagnetic pulses and/or said reduction of said deep tissue diathermy.

It is another object of the present invention to provide the methods as defined above, additionally comprising step of monitoring and/or controlling said steps of applying heat to a subcutaneous tissue within said region and/or said step of applying pulsed electromagnetic therapy to said region.

It is another object of the present invention to provide the methods as defined above, additionally comprising step of applying a dynamic magnetic field onto said region.

It is another object of the present invention to provide the methods as defined above, additionally comprising steps of
a. storing in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing electromagnetic radiation and heat radiation parameters selected from a group consisting of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. allowing said electromagnetic radiation and said heat radiation if parameters within said safe treatment parameters and to stop the electromagnetic radiation if the radiation parameters are in said unsafe treatment parameters.

It is another object of the present invention to provide the methods as defined above, additionally comprising step of selecting the shape of said electromagnetic pulse is selected in a non-limiting manner from a group consisting of square wave, a sine wave, a triangular wave, sawtooth wave, ramp waves, spiked wave or any other mathematical combination.

It is another object of the present invention to provide the methods as defined above, additionally comprising step of applying a triangular shaped electromagnetic pulse at frequency of 25 Hz and intensity of 20 Gauss.

It is another object of the present invention to provide the methods as defined above, additionally comprising step of applying a square shaped electromagnetic pulse at a frequency of 16 Hz, duration of about 5 milliseconds and intensity of 12 Gauss.

It is another object of the present invention to provide the methods as defined above, additionally comprising step of selecting said deep tissue diathermy device (4) from any device emitting RF radiation or any means adapted for producing electrical current absorbed by subcutaneous tissue.

It is another object of the present invention to provide the methods as defined above, wherein said step of applying heat to a subcutaneous tissue additionally comprising steps of
a. obtaining at least one electrical output device adapted to generate RF electromagnetic energy;
b. electrically coupling at least two electrodes to said electrical output device;
c. placing said at least two electrodes on said skin region; and,
d. simultaneously applying via all said electrodes said RF energy to said skin region.

It is another object of the present invention to provide the methods as defined above, wherein said step of applying heat to a subcutaneous tissue additionally comprising steps of
a. obtaining at least one at least one electrical output device adapted to generate electrical current;
b. electrically coupling at least two electrodes to said electrical output device;
c. placing said at least two electrodes on said skin region; and,
d. simultaneously applying via all said electrodes said electrical current to said skin region.

It is another object of the present invention to provide the methods as defined above, additionally comprising step of selecting said temperature T from a region of about 30 to about 80 degrees.

It is another object of the present invention to provide the methods as defined above, additionally comprising step of selecting the duration of each pulse applied by said pulsed electromagnetic frequency generator (2) to be higher than about 3 and lower than about 1000 milliseconds.

It is another object of the present invention to provide the methods as defined above, additionally comprising step of selecting the magnetic field intensity B of each pulse applied by said step of applying pulsed electromagnetic therapy to said region to be higher than about 0 and lower than about max magnetic field used in MM devices (i.e., 3 Tesla).

It is another object of the present invention to provide the methods as defined above, additionally comprising step of selecting the frequency F applied by the pulses applied by said step of applying pulsed electromagnetic therapy to said region to be higher than about 1 and lower than about 1M Hz.

It is another object of the present invention to provide the methods as defined above, additionally comprising step of selecting the energy E applied by said step of applying pulsed electromagnetic therapy to said region to be higher than about 1 and lower than about 150 watts per pulse.

It is another object of the present invention to provide the methods as defined above, wherein step of applying heat is applied for about 0.01 to 60 minutes.

It is another object of the present invention to provide the methods as defined above, wherein the heat and the pulsed electromagnetic therapy are applied simultaneously, sequentially or separately.

It is another object of the present invention to provide the methods as defined above, wherein said method is repeated 1 to 100 times in each treatment.

It is another object of the present invention to provide the methods as defined above, wherein said treatment is repeated more than once.

It is another object of the present invention to provide the methods as defined above, wherein said step of applying heat is performed by devices selected from a group consisting of: ultrasonic diathermy, an optical device, electromagnetic induction, sound waves emitting instrument, direct heat applying instrument, or from any other means of heating subcutaneous tissue to temperature T.

It is another object of the present invention to provide the methods as defined above, especially adapted to increase skin rejuvenation in the immediate (short) range.

It is another object of the present invention to provide the methods as defined above, especially adapted to increase skin rejuvenation in the intermediate range.

It is another object of the present invention to provide the methods as defined above, especially adapted to increase skin rejuvenation in the long range.

It is another object of the present invention to provide the methods as defined above, wherein said method is especially adapted to operate according to IEC selected from a group consisting of IEC 60601-2-35, IEC 60601-2-33, IEC 60601-2-29, IEC 60601-2-9, IEC 60601-2-5, IEC 60601-2-3, IEC 60601-1-8, IEC 60601-1-6, IEC 60601-1-4, IEC 60601-1-3, IEC 60601-1-2, IEC 60601-1-1 or any combination thereof.

It is another object of the present invention to provide an integrated system (20) adapted to increase skin rejuvenation of a region of a patient's skin, said system comprising at least two electrodes (41) adapted to be placed on said region of a patient's skin; each of said electrodes is at least partially coiled via a coil 42; wherein each of said electrodes is adapted for both (i) providing electromagnetic pulses to said region of a patient's skin; and, (ii) applying heat up to temperature T to said region of a patient's skin; further wherein all of said electrodes are adapted to simultaneously provide said electromagnetic pulses to said region of a patient's skin and apply heat to said region of a patient's skin.

It is another object of the present invention to provide the integrated system as defined above, wherein said heat applied to said region of a patient's skin is obtained by emitting RF radiation or via producing electrical current absorbed by subcutaneous tissue.

It is another object of the present invention to provide the integrated system as defined above, wherein application of said system increases said skin rejuvenation such that said skin rejuvenation increase (SRI) is greater than the sum of said SRI provided by electromagnetic pulses increase and said SRI provided by said deep tissue diathermy device increase.

It is another object of the present invention to provide the integrated system as defined above, wherein said electromagnetic pulse is a triangular shaped at frequency of 25 Hz and intensity of 20 Gauss.

It is another object of the present invention to provide the integrated system as defined above, wherein said electromagnetic pulse is square shaped at a frequency of 16 Hz, duration of about 5 milliseconds and intensity of 12 Gauss.

It is another object of the present invention to provide the integrated system as defined above, wherein said system reduces side effects and/or harmful effects of said electromagnetic pulses and/or said deep tissue diathermy such that said reduction of said side effects and/or said harmful effects is greater than the sum of said reduction of said electromagnetic pulses and/or said reduction of said deep tissue diathermy.

It is another object of the present invention to provide the integrated system as defined above, wherein said system additionally comprising a control system (6) adapted to regulate said electromagnetic pulses and/or said electromagnetic pulses.

It is another object of the present invention to provide the integrated system as defined above, wherein said system is adapted to provide a dynamic magnetic field that varies with time.

It is another object of the present invention to provide the integrated system as defined above, wherein the shape of said electromagnetic pulse is selected in a non-limiting manner from a group consisting of square wave, a sine wave, a triangular wave, sawtooth wave, ramp waves, spiked wave or any other mathematical combination.

It is another object of the present invention to provide the integrated system as defined above, wherein the magnetic field intensity B of each pulse applied by said pulsed electromagnetic frequency generator (2) ranges between about 0 and about 3 Tesla.

It is another object of the present invention to provide the integrated system as defined above, wherein the duration of each pulse applied by said system ranges between about 3 and about 1000 milliseconds.

It is another object of the present invention to provide the integrated system as defined above, wherein the frequency F applied by the pulses of said system ranges between about 1 Hz and about 1M Hz.

It is another object of the present invention to provide the integrated system as defined above, wherein the energy E applied by the pulses of said system ranges between about 1 and about 150 watts per pulse.

It is another object of the present invention to provide the integrated system as defined above, wherein said temperature T is higher than about 30 and lower than about 80 degrees.

It is another object of the present invention to provide the integrated system as defined above, wherein said power supply and control system (6) monitors physical tissue parameters and changes applied heat and electromagnetic pulses accordingly.

It is another object of the present invention to provide the integrated system as defined above, wherein said power supply and control system (6) additionally comprising:
a. processing means, adapted to store in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing means; adapted to sense electromagnetic radiation and heat radiation parameters selected from a group consisting of time t of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. regulating means, adapted to allow said electromagnetic radiation and heat radiation if said parameters are within said safe treatment parameters and to stop the electromagnetic radiation if the radiation parameters are in said unsafe treatment parameters.

It is another object of the present invention to provide the integrated system as defined above, wherein said power supply and control system (6) includes a mechanism for skin cooling.

It is another object of the present invention to provide the integrated system as defined above, especially adapted to increase skin rejuvenation in the immediate (short) range.

It is another object of the present invention to provide the integrated system as defined above, especially adapted to increase skin rejuvenation in the intermediate range.

It is another object of the present invention to provide the integrated system as defined above, especially adapted to increase skin rejuvenation in the long range.

It is another object of the present invention to provide the integrated system as defined above, wherein said system is especially adapted to operate according to IEC selected from a group consisting of IEC 60601-2-35, IEC 60601-2-33, IEC 60601-2-29, IEC 60601-2-9, IEC 60601-2-5, IEC 60601-2-3, IEC 60601-1-8, IEC 60601-1-6, IEC 60601-1-4, IEC 60601-1-3, IEC 60601-1-2, IEC 60601-1-1 or any combination thereof.

It is another object of the present invention to provide a method of increasing skin rejuvenation of a region of a patient's skin. The method comprises steps selected inter alia from:
a. obtaining an integrated system (20) adapted to increase skin rejuvenation; said integrated system (20) comprises: at least two electrodes (41) adapted to be placed on said region of a patient's skin; each of said electrodes is a least partially coiled via a coil 42;
b. applying heat to a subcutaneous tissue within said region up to temperature T whilst simultaneously applying pulses of electromagnetic field to said region;

wherein said increasing of said skin rejuvenation is greater than the sum of said applying heat to a subcutaneous tissue within said region and said applying pulses electromagnetic therapy to said region

It is another object of the present invention to provide the method as defined above, wherein said step of applying heat to a subcutaneous tissue within said region up to temperature T additionally comprising step of applying electrical current absorbed by subcutaneous tissue.

It is another object of the present invention to provide the method as defined above, wherein said system reduces [symbol] and/or [symbol] of said electromagnetic pulses and/or said deep tissue diathermy such that said reduction of said side effects and/or said harmful effects is greater than the sum of said reduction of said electromagnetic pulses and/or said reduction of said deep tissue diathermy.

It is another object of the present invention to provide the method as defined above, additionally comprising step of monitoring and/or controlling said steps of applying heat to a subcutaneous tissue within said region and/or said step of applying pulsed electromagnetic therapy to said region.

It is another object of the present invention to provide the method as defined above, additionally comprising step of applying a dynamic magnetic field onto said region.

It is another object of the present invention to provide the method as defined above, additionally comprising step of applying a triangular shaped electromagnetic pulse at frequency of 25 Hz and intensity of 20 Gauss.

It is another object of the present invention to provide the method as defined above, additionally comprising step of applying a square shaped electromagnetic pulse at a frequency of 16 Hz, duration of about 5 milliseconds and intensity of 12 Gauss.

It is another object of the present invention to provide the method as defined above, additionally comprising steps of
a. storing in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing electromagnetic radiation and heat radiation parameters selected from a group consisting of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. allowing said electromagnetic radiation and said heat radiation if parameters within said safe treatment parameters and to stop the electromagnetic radiation if the radiation parameters are in said unsafe treatment parameters.

It is another object of the present invention to provide the method as defined above, additionally comprising step of selecting the shape of said electromagnetic pulse is selected in a non-limiting manner from a group consisting of square wave, a sine wave, a triangular wave, sawtooth wave, ramp waves, spiked wave or any other mathematical combination.

It is another object of the present invention to provide the method as defined above, additionally comprising step of selecting said temperature T from a region of about 30 to about 80 degrees.

It is another object of the present invention to provide the method as defined above, additionally comprising step of selecting the duration of each pulse applied by said pulsed electromagnetic frequency generator (2) to be higher than about 3 and lower than about 1000 milliseconds.

It is another object of the present invention to provide the method as defined above, additionally comprising step of selecting the magnetic field intensity B of each pulse applied by said step of applying pulsed electromagnetic therapy to said region to be higher than about 0 and lower than about 3 Tesla.

It is another object of the present invention to provide the method as defined above, additionally comprising step of selecting the frequency F applied by the pulses applied by said step of applying pulsed electromagnetic therapy to said region to be higher than about 1 and lower than about 1M Hz.

It is another object of the present invention to provide the method as defined above, additionally comprising step of selecting the energy E applied by said step of applying pulsed electromagnetic therapy to said region to be higher than about 1 and lower than about 150 watts per pulse.

It is another object of the present invention to provide the method as defined above, wherein step of applying heat is applied for about 0.01 to 60 minutes.

It is another object of the present invention to provide the method as defined above, wherein the heat and the pulsed electromagnetic therapy are applied simultaneously, sequentially or separately.

It is another object of the present invention to provide the method as defined above, wherein said method is repeated 1 to 100 times in each treatment.

It is another object of the present invention to provide the method as defined above, wherein said treatment is repeated more than once.

It is another object of the present invention to provide the method as defined above, wherein said step of applying heat is performed by devices selected from a group consisting of: ultrasonic diathermy, an optical device, electromagnetic induction, sound waves emitting instrument, direct heat applying instrument, or from any other means of heating subcutaneous tissue to temperature T.

It is another object of the present invention to provide the method as defined above, especially adapted to increase skin rejuvenation in the immediate (short) range.

It is another object of the present invention to provide the method as defined above, especially adapted to increase skin rejuvenation in the intermediate range.

It is still an object of the present invention to provide the method as defined above, especially adapted to increase skin rejuvenation in the long range.

It is lastly an object of the present invention to provide the method as defined above, wherein said method is especially adapted to operate according to IEC selected from a group consisting of IEC 60601-2-35, IEC 60601-2-33, IEC 60601-2-29, IEC 60601-2-9, IEC 60601-2-5, IEC 60601-2-3, IEC 60601-1-8, IEC 60601-1-6, IEC 60601-1-4, IEC 60601-1-3, IEC 60601-1-2, IEC 60601-1-1 or any combination thereof.

### BRIEF DESCRIPTION OF THE DRAWINGS

In order to understand the invention and to see how it may be implemented in practice, a few preferred embodiments will now be described, by way of non-limiting example only, with reference to the accompanying drawings, in which:

FIGS. 1A-1D schematically present a skin viability improving system (10), comprising a pulsed electromagnetic frequency generator (2) and a deep tissue diathermy device (4).

FIG. 1E is a diagram illustrating an example of electrical current applied by the deep tissue diathermy device (4). The current involves a maximal amplitude of 160 volts, and a frequency close to 1 Hz.

FIG. 2 schematically presents a skin viability improving system (10), comprising a pulsed electromagnetic frequency generator (2), a deep tissue diathermy device (4) and a power supply and control system (6).

FIG. 3 schematically presents square waves at a rate of 16 Hz in duration of about 5 milliseconds in an intensity of 12 Gauss which stimulate angiogenesis.

FIG. 4 schematically presents triangular wave pulses at a frequency of 25 Hz and intensity of 20 Gauss which stimulate collagen production.

FIGS. 5-6 illustrate another embodiment of the skin viability improving system (20).

FIGS. 7-12 schematically present the methods of improving skin viability (400-420).

FIGS. 13A1-13C2 are pictures of one patient out of the study group treated with the device of the present invention.

FIGS. 14A1-14C2 are pictures of one patient out of the first control group treated with the device of the present invention on the right side and RF on the left side.

FIGS. 15A1-15C2 are pictures of one patient out of the second control group treated with the device of the present invention on the right side and PEMF on the left side.

FIGS. 16A1-16C2 are pictures of one patient out of the third control group treated with the device of the present invention on the right side. The left side was treated with PEMF followed by RF.

FIG. 17 illustrates another embodiment of the device of the present invention.

FIG. 18 illustrates a cyclic diagram according to an embodiment.

### DETAILED DESCRIPTION OF THE DRAWINGS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide means and method for increasing the viability of the skin. Yet more the present invention provides means and system for skin tightening and rejuvenation.

It is one object of the present invention to disclose a device used to improve skin viability, by a synergistic approach of deep tissue diathermy combined with application of PEMF, wherein at least two devices of deep tissue diathermy are incorporated, one of them based on PEMF therapy. The latter improves the healing process initiated by the at least one other device of deep tissue diathermy.

The term “Pulsed Electromagnetic Fields (PEMF)” refers hereinafter in a non-limiting manner to electromagnetic radiation of different frequencies—ranging from static magnetic fields, through extremely low frequencies (ELF) to radiofrequencies (RF)—administered in pulses.

The term “Radio Frequency (RF)” refers hereinafter in a non-limiting manner to part of the electromagnetic spectrum with frequency range of about 3 Hz to 300 GHz.

The term “Extremely Low Frequencies (ELF)” refers hereinafter in a non-limiting manner to part of the RF electromagnetic spectrum with frequency range of about 3 Hz to 30 GHz

The term “collagen” refers hereinafter in a non-limiting manner to a long, fibrous structural protein which is a major component of the extracellular matrix that supports most tissues and gives cells structure. It is responsible for skin strength and elasticity, and its degradation leads to wrinkles that accompany aging.

The term “epidermis” refers hereinafter in a non-limiting manner to the outermost layer of the skin.

The term “dermis” refers hereinafter in a non-limiting manner to a layer of skin beneath the epidermis that consists of connective tissue, and cushions the body from stress and strain.

The term “deep tissue diathermy” refers hereinafter in a non-limiting manner to a device which heats tissues beneath the epidermis.

The term “electric diathermy” refers hereinafter in a non-limiting manner to a device which uses high frequency alternating electric or magnetic fields, sometimes with no electrode or device contact to the skin, to induce gentle deep tissue heating by induction. For collagen fiber stimulation, typical electrical parameters may include, in a non limiting manner, frequency of about 1 MHz, energy of about 80 joule per 1 square tissue volume, applied for about 6 seconds.

The term “ultrasonic diathermy” refers hereinafter in a non-limiting manner to heating of tissues by ultrasound.

The term “about” refers hereinafter to a range of 25% below or above the referred value.

The term “physical tissue parameters” refers hereinafter to parameters such as tissue temperature, electric current, tissue impedance, specific absorption rate (SAR), treatment depth and superficial muscle contractions.

The term “angiogenesis” refers hereinafter to the sprouting of new blood vessels.

The term “square wave” refers hereinafter to a non-sinusoidal waveform named for its triangular shape.

The term “triangle wave” refers hereinafter to a non-sinusoidal waveform named for its triangular shape.

The term “International Electrotechnical Commission Standards (IEC) 60601-1” refers hereinafter to a medical electrical equipment standard. More specifically it refers to general requirements for basic safety and essential performance.

Consistent with the notation above, the term “IEC 60601-1-1” refers hereinafter to a medical electrical equipment standard. More specifically it refers to general requirements for safety—Collateral standard: Safety requirements for medical electrical systems. The IEC 60601-1 set of standards are divided into three distinct areas. The first area is the basic standard IEC 60601-1. This is the general requirement for all electrical medical based products. The second area is the collateral standards, which cover across the board issues such as combining into a system with other devices, EMC, radiation protection, and programmable electronic medical systems (software, firmware, etc.). The standard numbers are IEC 60601-1-1, -1-2, -1-3, and -1-4 respectively. The third area is the particular standards that deal with a specific type of medical device. The particular standards are identified as IEC 60601-2-XX where XX identifies the particular standard number for the particular type of medical equipment. An example would be IEC 60601-2-3 which is the particular standard for short-wave therapy equipment.

The term “IEC 60601-1-2” refers hereinafter to a medical electrical equipment standard. More specifically it refers to general requirements for basic safety and essential performance—Collateral standard: Electromagnetic compatibility—Requirements and tests.

The term “IEC 60601-1-3” refers hereinafter to a medical electrical equipment standard. More specifically it refers to general requirements for basic safety and essential performance—Collateral Standard: Radiation protection in diagnostic X-ray equipment.

The term “IEC 60601-1-4” refers hereinafter to a medical electrical equipment standard. More specifically it refers to general requirements for safety—Collateral Standard: Programmable electrical medical systems.

The term “IEC 60601-1-6” refers hereinafter to a medical electrical equipment standard. More specifically it refers to general requirements for basic safety and essential performance—Collateral standard: Usability.

The term “IEC 60601-1-8” refers hereinafter to a medical electrical equipment standard. More specifically it refers to general requirements for basic safety and essential performance—Collateral Standard: General requirements, tests and guidance for alarm systems in medical electrical equipment and medical electrical systems.

The term “IEC 60601-2-3” refers hereinafter to a medical electrical equipment standard. More specifically it refers to particular requirements for the safety of short-wave therapy equipment.

The term “IEC 60601-2-5” refers hereinafter to a medical electrical equipment standard. More specifically it refers to particular requirements for the safety of ultrasonic physiotherapy equipment.

The term “IEC 60601-2-9” refers hereinafter to a medical electrical equipment. More specifically it refers to particular requirements for the safety of patient contact dosemeters used in radiotherapy with electrically connected radiation detectors.

The term “IEC 60601-2-29” refers hereinafter to a medical electrical equipment standard. More specifically it refers to particular requirements for the basic safety and essential performance of radiotherapy simulators.

The term “IEC 60601-2-33” refers hereinafter to a medical electrical equipment standard. More specifically it refers to particular requirements for the safety of magnetic resonance equipment for medical diagnosis.

The term “IEC 60601-2-35” refers hereinafter to a medical electrical equipment standard. More specifically it refers to particular requirements for the safety of blankets, pads and mattresses intended for heating in medical use.

The present invention relates to a physical therapeutic methods and systems. In said systems a dynamic magnetic pulse and electromagnetic heating systems are incorporated together to accomplish a physical therapy, epically skin tightening and rejuvenation.

The present invention provides a system adapted to increase skin rejuvenation of a region of a patient's skin. The system comprising in a non-limiting manner the following:

a. a pulsed electromagnetic field (PEMF) frequency generator (2) for constantly providing electromagnetic pulses to said region of a patient's skin; and,
b. a deep tissue diathermy device (4) applying heat to said region of a patient's skin up to temperature T;

The system (10) is adapted for simultaneously apply heat and PEMF to said region of a patient's skin. Furthermore, the system increases the skin rejuvenation such that the increase is greater than the sum of the electromagnetic pulses increase and the deep tissue diathermy.

Furthermore the system reduces [symbol] and/or [symbol] of the electromagnetic pulses and/or said deep tissue diathermy such that said reduction of said side effects and/or said harmful effects is greater than the sum of said reduction of said electromagnetic pulses and/or said reduction of said deep tissue diathermy.

It is another object of the present invention to provide an integrated system (20) adapted to increase skin rejuvenation of a region of a patient's skin. The system comprising at least two electrodes adapted to be placed on said region of a patient's skin; each of said electrodes is at least partially coiled via a coil. It is emphasized that each of said electrodes is adapted for both (i) providing electromagnetic pulses to said region of a patient's skin; and, (ii) applying heat up to temperature T to said region of a patient's skin. Furthermore, it is emphasized that all of said electrodes are adapted to simultaneously provide electromagnetic pulses to said region of a patient's skin; and, apply heat up to temperature T to said region of a patient's skin.

The decrease in the side effects of the deep tissue diathermy by the healing effect of the pulsed electromagnetic frequency therapy.

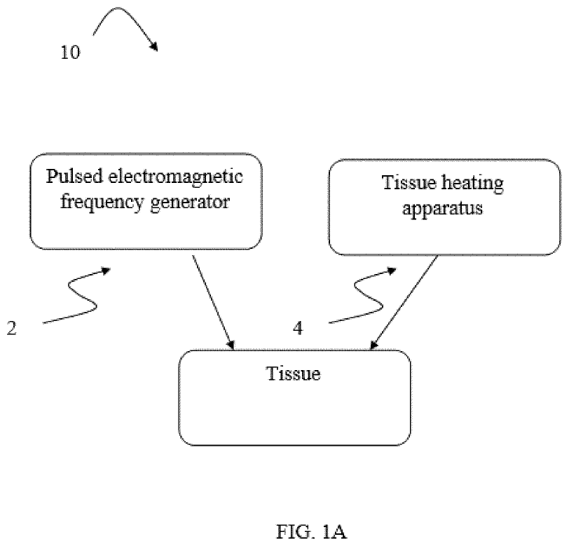

Reference is now made to FIGS. 1A-1D, illustrating the system 10 for increasing skin rejuvenation. As described above, the system comprising a pulsed electromagnetic frequency generator (2) for providing electromagnetic pulses to the region of a patient's skin; and, a deep tissue diathermy device (4) adapted to apply heat to the region of a patient's skin up to temperature T.

It is emphasized that the system increases the skin rejuvenation such that the increase is greater than the sum of the electromagnetic pulses increase and the deep tissue diathermy.

By exposing the tissue (a region of a patient's skin) to the combination of regulated heat and a pulsed electromagnetic filed a synergic effect of improving skin rejuvenation is obtained.

The present invention relays on 2 effects, the thermal effect and the electromagnetic pulse effect:

The thermal effect includes heating the tissue such that when the tissue is heated to a sufficiently high temperature, tissue injury is produced. Furthermore, when heat is generated within the dermis, it usually causes contraction and thickening of collagen fibers. This results in an overall tightened and rejuvenated appearance of the skin.

Heat within the dermis creates a limited thermal injury. The body's natural response to this injury is to produce collagen at the site of the wound. This results in firmer, thicker, more youthful skin. Usually the skin is heated to temperatures below 60 degrees for short periods of time. The thermal effect i.e., can be produced by:
1. Optical means (e.g. an optical emitter, an illuminator, etc.)—by emitting light in different wavelengths absorbed by subcutaneous tissue such that said tissue is heated; or
2. Electrical means (e.g., current circuitry, a current generator)—by passing electrical current; or
3. Electromagnetic means (e.g., an electromagnetic emitter, electromagnetic circuitry, induction circuitry, etc.)—by transmitting or inducting (electromagnetic induction) electromagnetic filed on the skin; or
4. Sound waves (e.g., an acoustic emitter)—specifically in the ultrasound frequencies; or
5. Physical means (e.g., an applicator such as a massager, a topical application)—such as massage or applying warm substance adjacent to the skin; or any combination thereof.

The electromagnetic pulses (either dynamic or static) may start natural healing processes which usually occur in response to an injury (especially, angiogenesis, and generation of new collagen fibers via the release of tissue growth factors).

Said electromagnetic field generates movements of charged molecules (ions) within the inter cellular fluids. This movement generates heat which may enhance the thermal effect discussed earlier.

It is acknowledged that healing is the process by which the cells in the body regenerate and repair to reduce the size of a damaged area. Healing incorporates both the removal of necrotic tissue (demolition), and the replacement of this tissue.

The replacement can happen in two ways:
1. by regeneration: the necrotic cells are replaced by the same tissue as was originally there.
2. by repair: injured tissue is replaced with scar tissue.

The Pulsed Electromagnetic Fields (PEMF) applied by the system 10, as described above, has no thermal effects. Said no thermal effects rely on the tissue components and their reaction to the applied radiation characteristics. These effects might be due to the reaction of large charged molecules and various frequencies and frequency harmonies, charged small ions in the cell membranes affecting the cells function and reactions to hormones and chemical signals, charged small ions in the extracellular space and other purely understood mechanisms.

Furthermore, applying the radiation in pulses was also found to have non thermal effects. Yet more, only a specific combination of frequency, duty cycle and transmitted power achieve a specific tissue response.

It is now commonly accepted that electromagnetic fields (EMF) or PEMF are capable of initiating various healing processes and for treatment of pain and edema in superficial soft tissues two decades ago. [Rosch, P. J., Markov, M. S., eds. Bioelectromagnetic Medicine, Marcel Dekker, NY, 251-264].

The present invention utilizes PEMF (combined with heat applying source) for cosmetic purposes as described above. The important role of PEMF in the specific field intensities and frequencies increases epidermal collagen synthesis. This new formed collagen increases skin elasticity and rejuvenates its appearance. Furthermore, PEMF increases the degree of endothelial cell tubulization and proliferation, and augmented angiogenesis primarily by stimulating endothelial release of FGF-2, inducing paracrine and autocrine changes in the surrounding tissue. Angiogenesis, the sprouting of new blood vessels, increases blood flow to the tissue, which in turn increases oxygen and nutritional substances delivery to the tissue. This effect is most beneficial for an injured tissue, promoting rapid and improved healing. The growth factor released further enhances the healing process, both in quality and time of improvement.

The following disclosure is a more detailed description of the two combined effects.

As disclosed earlier, the present invention discloses a system 10 which incorporates both regulated heating means (a heater) and electromagnetic pulses.

As described above, the heat can be produced by:
1. Optical means—by emitting light in different wavelengths absorbed by subcutaneous tissue such that said tissue is heated (e.g., from an illuminator, such as an array of LEDs).
2. Electrical means—by passing electrical current.
3. Electromagnetic means—by transmitting or inducting (electromagnetic induction) electromagnetic filed on the skin.
4. Sound waves—specifically in the ultrasound frequencies.
5. Physical means—such as massage or applying warm substance adjacent to the skin.

Reference is now made to FIGS. 1B-1D, illustrating the system 10 according to preferred embodiment of the present invention.

According to a preferred embodiment of the present invention, the deep tissue diathermy device (4) comprises:
a. at least one electrical output device adapted to generate electrical current; and,
b. at least two electrodes (41) electrically coupled to said electrical output device and placed on said skin region.

According to said embodiment all said electrodes are adapted to simultaneously apply said electrical current to said skin region.

FIG. 1B illustrates system 10 in which the deep tissue diathermy device (4) comprises 4 electrodes (denoted by numerical reference 41).

FIGS. 1C-1D illustrate the system 10 in which the deep tissue diathermy device (4) comprises 8 electrodes (denoted by numerical reference 41).

Reference is now made to FIG. 1E illustrating an example of electrical current applied by the deep tissue diathermy device (4). The current involves a maximal amplitude of 160 volts, and a frequency close to 1 Hz.

According to another embodiment of the present invention, the pulsed electromagnetic frequency generator is adapted to provide an electromagnetic field which varies with time (dynamic magnetic field).

According to another embodiment of the present invention, the pulsed electromagnetic frequency generator (2) which provides electromagnetic pulses to the patient's skin is positioned near the treated tissue and emits a dynamic magnetic field which varies with time. The dynamic magnetic field can vary according to any specific treatments. For examples, to stimulate angiogenesis, pulses at a frequency of 16 Hz, intensity of 12 Gauss and duration of about 5 milliseconds are generated. Alternatively, to stimulate collagen production a triangular wave pulses at a frequency of 25 Hz and intensity of 20 Gauss are generated.

The deep tissue diathermy device (4) is adapted to apply heat to said region of a patient's skin up to temperature T. According to one embodiment of the present invention the heat is applied by passing electrical current through the tissue. The electrical current can be performed in one of the following three manners:
1. Through at least one electrode which is in direct physical contact with the skin;
2. through at least one electrode which is not in physical contact with the skin, and the electrical current is transferred by induction.
3. through at least one antenna which passes the electrical current to the skin via electromagnetic induction.

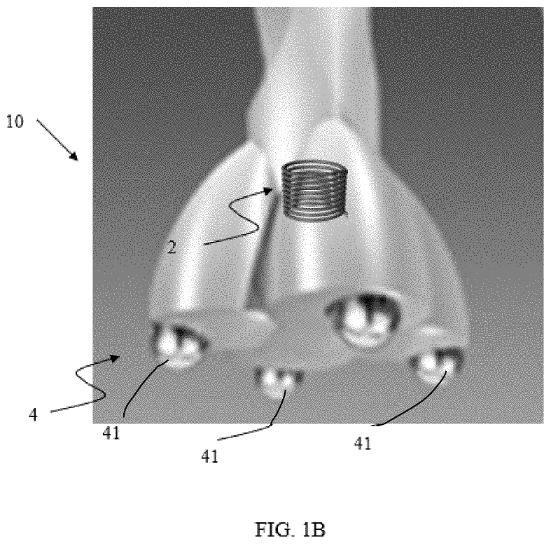

Reference is now made to FIG. 2, which illustrates another embodiment of the present invention, According to that embodiment the system additionally comprising a control system (6) adapted to regulate said electromagnetic pulses and/or said electromagnetic pulses.

According to another embodiment of the present invention the treatment is provided only in safe treatment parameters.

Safe treatment parameters are defined by the parameters in table 1:

TABLE 1
safe treatment parameters

| parameter | Values |
|---|---|
| Time, t | 0-600 Minutes |
| Temperature, T | 25-80 Celsius |
| Duty cycle t/T | 0-100% |
| Frequency MHz | DC-10 Mhz |

Unsafe safe treatment parameters are defined by the parameters in table 2:

TABLE 2
unsafe treatment parameters

| parameter | Values | |
|---|---|---|
| power P | 0-100 | Watt |
| | >10 | hours |
| | (none stop) | |
| [symbol] E | 0-200 | Jowls |
| magnetic field | 0-10 | Tesla |
| | >80 | Celsius |
| [symbol] Bre | 30 | Millimeters |
| [symbol] epth D of said | | |
| treated tissue | | |
| Duty cycle t/T | N/A | |
| Frequency | >10 | MHz |
| MHz | | |
| power P | >100 | Watt |
| Energy E | >200 | Jowls |
| magnetic field | >10 | Tesla |
| intensity B | | |
| Depth D of | >30 | Millimeter |

said treated
tissue

According to another embodiment the, control system (6) additionally comprises:
(a) processing means, adapted to store in a communicable database predetermined parameters defining (i) safe treatment parameters and (ii) unsafe treatment parameters; said parameters are selected from a group consisting of time t of said treatment, temperature T of said tissue, duty cycle t/T, Frequency F, power P, Intensity I of said ultrasound diathermy, energy E applied by the pulses of said pulsed electromagnetic frequency generator, treatment depth D of said treated tissue, magnetic field intensity B, tissue impedance, specific absorption rate (SAR), superficial muscle contractions or a combination thereof;
(b) sensing means; adapted to sense electromagnetic radiation and heat radiation parameters selected from a group consisting of time t of time t of said treatment, temperature T of said tissue, duty cycle t/T, Frequency F, power P, Intensity I of said ultrasound diathermy, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
(c) regulating means, adapted to allow said pulsed electromagnetic radiation and heat radiation if said parameters are within said safe treatment parameters and to stop the pulsed electromagnetic radiation if the radiation parameters are in said unsafe treatment parameters.

According to another embodiment, the system as defined above, additionally comprising sensors for monitoring physical parameters selected form a group consisting of time t of said treatment, temperature T of said tissue, duty cycle t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof.

The sensors receives said parameters from the treated tissue and changes the parameters of the pulsed electromagnetic frequency generator (2) and the deep tissue diathermy device (4) to optimize the effect of each component and/or to augment the synergistic effect of both components, whilst avoiding harm to the tissue.

According to another embodiment of the present invention the shape of the electromagnetic pulse is selected in a non-limiting manner from a group consisting of square wave, a sine wave, a triangular wave, sawtooth wave, ramp waves, spiked wave or any other mathematical combination.

According to another embodiment, the system as defined above is adapted to provide electromagnetic pulse at a frequency of 16 Hz which increases from 0 Gauss to 12 Gauss.

According to another embodiment, the system as defined above is adapted to provide electromagnetic square wave pulse at a frequency of 16 Hz which increases from 0 Gauss to 12 Gauss.

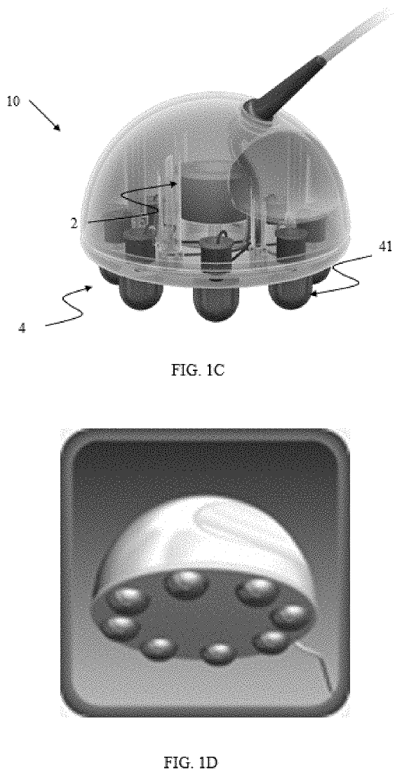

According to another embodiment, the system as defined above is adapted to provide short square waves at a rate of 16 Hz in duration of about 5 milliseconds in an intensity of 12 Gauss. Such square wave pulses are especially provided to stimulate angiogenesis. Reference is now made to FIG. 3, which illustrates such square wave pulse.

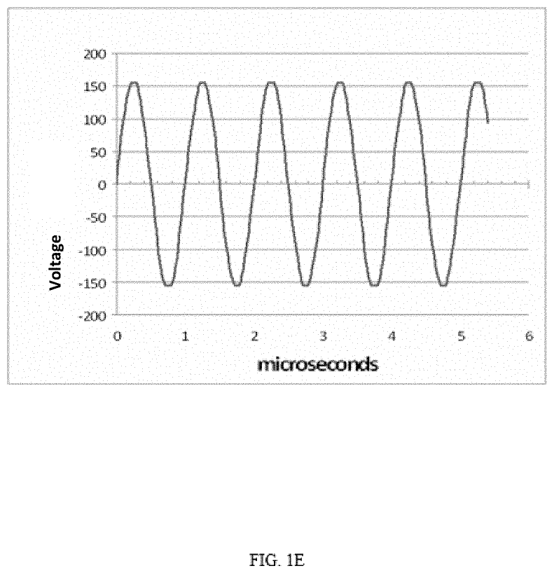

According to another embodiment, the system as defined above is adapted to provide triangular wave pulses at a frequency of 25 Hz and intensity of 20 Gauss. Such pulses are especially provided to stimulate collagen production. Reference is now made to FIG. 4, which illustrates such triangular wave pulses.

According to another embodiment, the system as defined above is adapted to provide alternating current (AC) at a frequency of 1 MHz.

According to another embodiment, the system as defined above is adapted to provide intensity of about 80 J/cm² sec.

According to another embodiment of the present invention the magnetic field intensity B of each pulse applied by said pulsed electromagnetic frequency generator (2) ranges between about 0 and about the max magnetic field used in MM devices (i.e., 3 Tesla).

According to another embodiment of the present invention, the duration of each pulse applied by said pulsed electromagnetic frequency generator (2) ranges between about 3 and about 1000 milliseconds.

According to another embodiment of the present invention, the frequency F applied by the pulses of said pulsed electromagnetic frequency generator (2) ranges between about 1 and about 1M Hz.

According to another embodiment of the present invention, the energy E applied by the pulses of said pulsed electromagnetic frequency generator (2) ranges between about 1 and about 150 watts per pulse.

According to another embodiment of the present invention, the deep tissue diathermy device (4) is selected in a non-limiting manner from a group consisting of electric diathermy or any device emitting RF radiation absorbed by subcutaneous tissue.

According to another embodiment of the present invention, the deep tissue diathermy device (4) is selected in a non-limiting manner from a group consisting of an ultrasonic diathermy, an optical device, electromagnetic induction, sound waves emitting instrument, direct heat applying instrument, or from any other means of heating subcutaneous tissue to temperature T.

According to another embodiment of the present invention, the optical device is adapted to emit light in wavelengths absorbed by subcutaneous tissue such that said subcutaneous tissue is heated.

According to another embodiment of the present invention, the sound waves emitting instrument is adapted to emit sound waves absorbed by the subcutaneous tissue such that said subcutaneous tissue is heated.

According to another embodiment of the present invention, the temperature T is higher than about 30 and lower than about 80 degrees.

According to another embodiment of the present invention, the power supply and control system (6) includes a mechanism for skin cooling.

According to another embodiment of the present invention, system (10) is encased in at least one platform.

According to another embodiment of the present invention, the pulsed electromagnetic frequency generator (2) and said deep tissue diathermy device (4) have more than one applicator to treat more than one body part simultaneously.

According to another embodiment of the present invention, the pulsed electromagnetic frequency generator (2) has electrostatic shielding.

Is should be emphasized that the system as defined in any of the embodiments produces synergic outcomes in the following three ranges:

The immediate (short) range, in the intermediate range and in the long range.

In the immediate (short) range—the contraction and thickening of collagen fibers occur, which in turn results in an overall tightened and rejuvenated appearance of the skin.

In the intermediate range (i.e., about two to three weeks)—new epidermal cells and new collagen fibers are produced.

In the long range (i.e., about a month)—the cellulite is scattered.

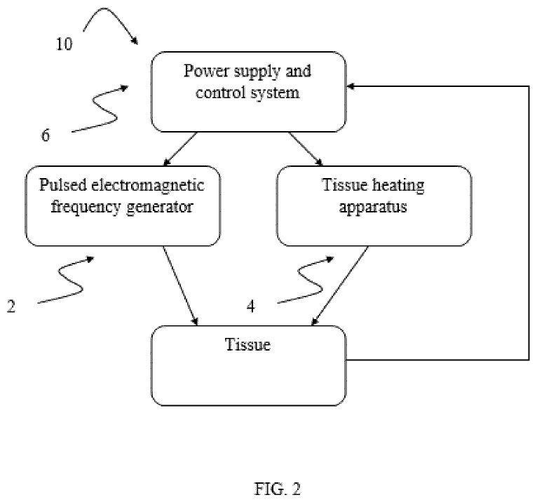

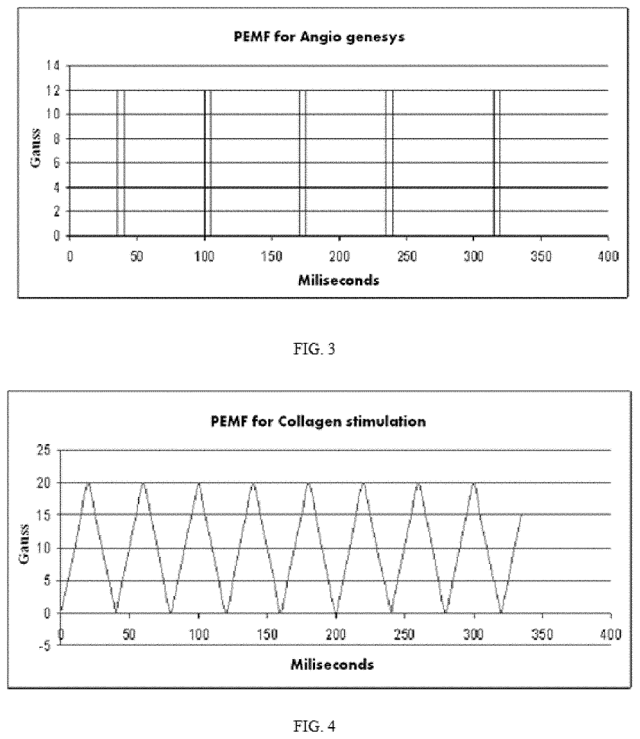

Reference is now made to FIGS. 5-6 illustrating another system (20) according to a preferred embodiment of the present invention. According to this embodiment, an integrated system (20) adapted to increase skin rejuvenation of a region of a patient's skin is provided.

The system 20 comprising at least two electrodes (41) adapted to be placed on said region of a patient's skin; each of said electrodes is a least partially coiled (or looped) via a coil (42).

It should be emphasized that each of said electrodes is adapted for both (i) providing electromagnetic pulses to said region of a patient's skin (via said coil); and, (ii) applying heat up to temperature T to said region of a patient's skin.

Furthermore, it should be emphasized that all said electrodes provide simultaneously electromagnetic pulses to said region of a patient's skin; and, apply heat up to temperature T to said region of a patient's skin.

The heat is provided to the skin by applying electrical current through said electrodes which is absorbed by subcutaneous tissue.

FIG. 5 illustrates such a system 20 comprising 4 electrodes (denotes as 41) and FIG. 6 illustrates such a system comprising 8 electrodes (denotes as 41).

It should be emphasized that the application of said system 20 increases said skin rejuvenation such that said skin rejuvenation increase (SRI) is greater than the sum of said SRI provided by electromagnetic pulses increase and said SRI provided by said deep tissue diathermy device increase.

According to another embodiment of the present invention the electromagnetic pulse in system 20 is a triangular shaped at frequency of 25 Hz and intensity of 20 Gauss.

According to another embodiment of the present invention the electromagnetic pulse in system 20 is square shaped at a frequency of 16 Hz, duration of about 5 milliseconds and intensity of 12 Gauss.

According to another embodiment of the present invention, the system 20 reduces side effects and/or harmful effects of said electromagnetic pulses and/or said deep tissue diathermy such that said reduction of said side effects and/or said harmful effects is greater than the sum of said reduction of said electromagnetic pulses and/or said reduction of said deep tissue diathermy.

According to another embodiment of the present invention, the system 20 additionally comprising a control system (6) adapted to regulate said electromagnetic pulses and/or said electromagnetic pulses.

According to another embodiment of the present invention, the system is adapted to provide a dynamic magnetic field that varies with time.

According to another embodiment of the present invention, the shape of said electromagnetic pulse in system 20 is selected in a non-limiting manner from a group consisting of square wave, a sine wave, a triangular wave, sawtooth wave, ramp waves, spiked wave or any other mathematical combination.

According to another embodiment of the present invention, the magnetic field intensity B in system 20 of each pulse applied by said pulsed electromagnetic frequency generator (2) ranges between about 0 and about 3 Tesla.

According to another embodiment of the present invention, the duration of each pulse applied in system 20 ranges between about 3 and about 1000 milliseconds.

According to another embodiment of the present invention, the frequency F applied by the pulses of said system ranges between about 1 Hz and about 1M Hz.

According to another embodiment of the present invention, the energy E applied by said system ranges between about 1 and about 150 watts per pulse.

According to another embodiment of the present invention, the temperature T is higher than about 30 and lower than about 80 degrees.

According to another embodiment of the present invention, the power supply and control system (6) in system (20) monitors physical tissue parameters and changes applied heat and electromagnetic pulses accordingly.

According to another embodiment of the present invention, the power supply and control system (6) additionally comprising:
a. processing means (e.g., a processor configured to communicate with a non-transitory computer readable medium configured to store instructions thereon which are executable by the processor to cause the processor to carry out operations), adapted to store in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing means (e.g., a sensor, a radiation sensor, a thermal sensor, a combination sensor, etc.); adapted to sense electromagnetic radiation and heat radiation parameters selected from a group consisting of time t of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. regulating means (e.g., a regulator, a controller, control processing or regulator processing carried out by the above-noted processor), adapted to allow said electromagnetic radiation and heat radiation if said parameters are within said safe treatment parameters and to stop the electromagnetic radiation if the radiation parameters are in said unsafe treatment parameters.

According to another embodiment of the present invention, the power supply and control system (6) includes a mechanism for skin cooling.

According to another embodiment of the present invention, the system (20) is especially adapted to increase skin rejuvenation in the immediate (short) range.

According to another embodiment of the present invention, the system (20) is especially adapted to increase skin rejuvenation in the intermediate range.

According to another embodiment of the present invention, the system (20) is especially adapted to increase skin rejuvenation in the long range.

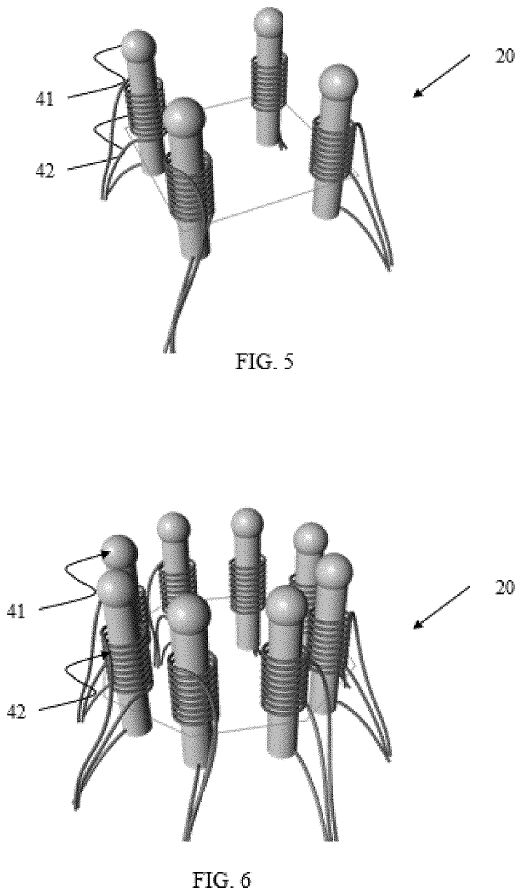

Reference is now made to FIG. 7, schematically illustrating one possible method (400) of increasing skin rejuvenation of a region of a patient's skin. The method comprising steps selected inter alia from obtaining (i) pulsed electromagnetic frequency generator; and, (ii) a deep tissue diathermy device (401); applying heat to a subcutaneous tissue within said region up to temperature T (402); said temperature T is optimized for production of new dermal ground substance and collagen contraction. While the collagen contraction tightens the skin and conceals wrinkles immediately, the dermal proliferation and new collagen production has a later effect. The next step is applying additional pulsed electromagnetic field (403) which generates a healing mechanism of the heated skin, which includes growth factor and cytokines release and eventually angiogenesis.

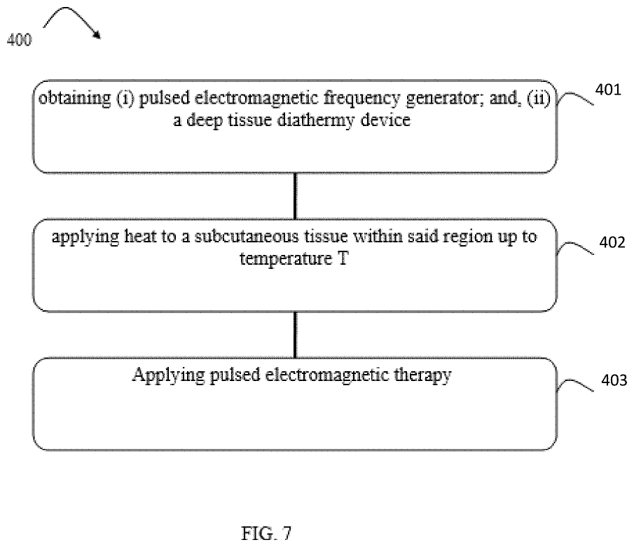

Reference is now made to FIG. 8, which illustrates another preferred method of the present invention. According to this embodiment, the method 400 additionally comprises the step of: monitoring and/or controlling said steps of applying heat to a subcutaneous tissue within said region and/or said step of applying pulsed electromagnetic therapy to said region (404).

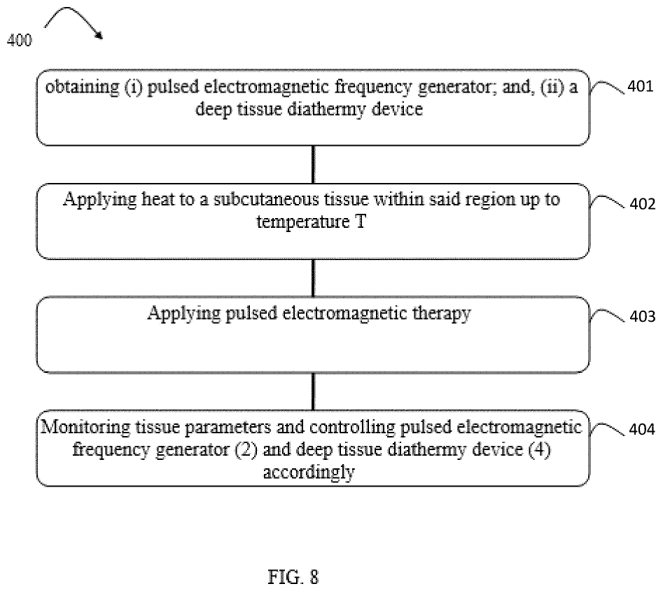

Reference is now made to FIG. 9, schematically illustrating one possible method (410) of increasing skin rejuvenation of a region of a patient's skin. The method comprising steps selected inter alia from obtaining (i) pulsed electromagnetic frequency generator; and, (ii) a deep tissue diathermy device (411). The next step is applying additional pulsed electromagnetic field (412) which generates a healing mechanism of the heated skin, which includes growth factor and cytokines release and eventually angiogenesis. The final step is applying heat to a subcutaneous tissue within said region up to temperature T (413); said temperature T is optimized for production of new dermal ground substance and collagen contraction. While the collagen contraction tightens the skin and conceals wrinkles immediately, the dermal proliferation and new collagen production has a later effect.

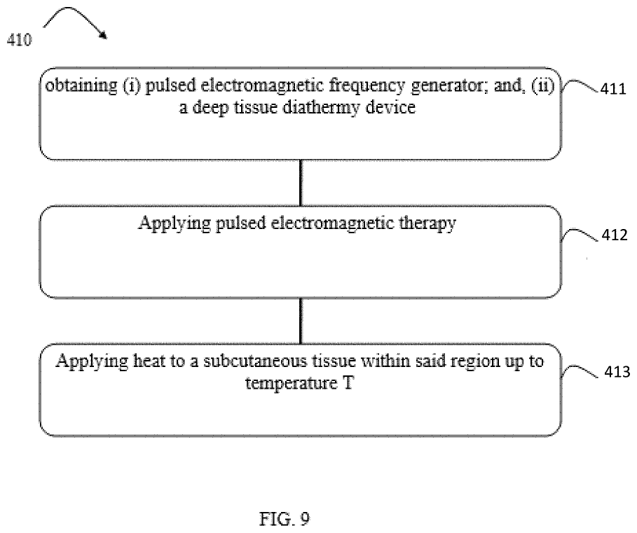

Reference is now made to FIG. 10, which illustrates another preferred method of the present invention. According to this embodiment, the method 410 additionally comprises the step of: monitoring and/or controlling said steps of applying heat to a subcutaneous tissue within said region and/or said step of applying pulsed electromagnetic therapy to said region (414).

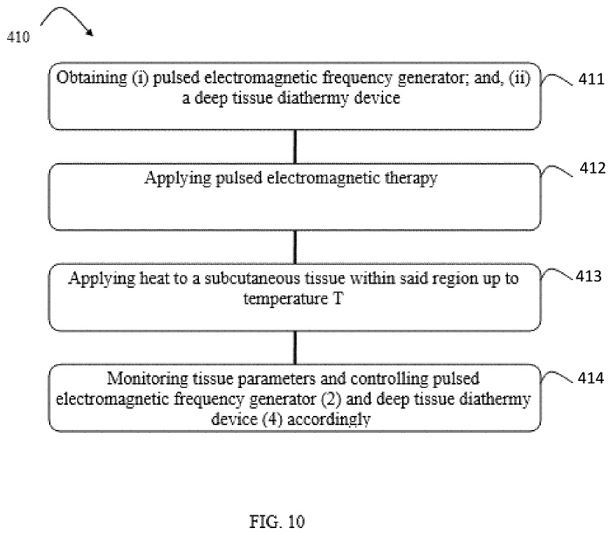

Reference is now made to FIG. 11, schematically illustrating one possible method (420) of increasing skin rejuvenation of a region of a patient's skin. The method comprising steps selected inter alia from obtaining (i) pulsed electromagnetic frequency generator; and, (ii) a deep tissue diathermy device (421). The next step is applying additional pulsed electromagnetic field (422) whilst simultaneously applying heat to a subcutaneous tissue within said region up to temperature T. The electromagnetic pulses generate a healing mechanism of the heated skin, which includes growth factor and cytokines release and eventually angiogenesis. The heat applied temperature T is optimized for production of new dermal ground substance and collagen contraction. While the collagen contraction tightens the skin and conceals wrinkles immediately, the dermal proliferation and new collagen production has a later effect.

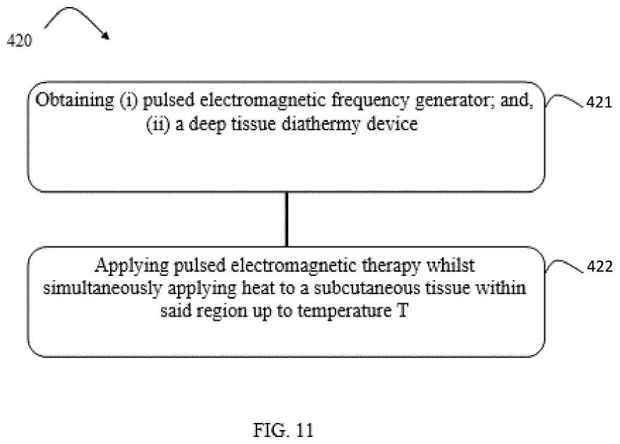

Reference is now made to FIG. 12, which illustrates another preferred method of the present invention. According to this embodiment, the method 420 additionally comprises the step of: monitoring and/or controlling said steps of applying heat to a subcutaneous tissue within said region and/or said step of applying pulsed electromagnetic therapy to said region (424).

According to another embodiment of the present invention, each of the methods as defined above additionally comprising step of selecting said temperature T from a region of about 30 to about 80 degrees.

According to another embodiment of the present invention, each of the methods as defined above additionally comprising step of applying a dynamic magnetic field onto said region.

According to another embodiment of the present invention, each of the methods as defined above additionally comprising steps of:
a. storing in a communicable database predetermined parameters defining (i) safe treatment parameters and (ii) unsafe treatment parameters; said parameters are selected from a group consisting of time t of said treatment, temperature T of said tissue, duty cycle t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
b. sensing electromagnetic radiation and heat radiation parameters selected from a group consisting of time t of time t of said treatment, temperature T of said tissue, duty cycle t/T, Frequency F, power P, Intensity I of said ultrasound diathermy, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof;
c. allowing said electromagnetic radiation and said heat radiation if parameters are within said safe treatment parameters and stopping the electromagnetic radiation if the radiation parameters are in said unsafe treatment parameters.

According to another embodiment of the present invention, the step of applying heat is performed by devices selected from a group consisting of: ultrasonic diathermy, an optical device, electromagnetic induction, sound waves emitting instrument, direct heat applying instrument, or from any other means of heating subcutaneous tissue to temperature T.

According to another embodiment of the present invention, each of the methods as defined above additionally comprising step of selecting the magnetic field intensity B of each pulse applied by said step of applying pulsed electromagnetic therapy to said region to be higher than about 0 and lower than about max magnetic field used in Mill devices (i.e., 3 Tesla).

According to another embodiment of the present invention, each of the methods as defined above additionally comprising step of selecting the frequency F applied by the pulses applied by said step of applying pulsed electromagnetic therapy to said region to be higher than about 1 Hz and lower than about 1 MHz.

According to another embodiment of the present invention, each of the methods as defined above additionally comprising step of selecting the energy E applied by said step of applying pulsed electromagnetic therapy to said region to be higher than about 1 and lower than about 150 watts per pulse.

According to another embodiment of the present invention, in each of the methods as defined above the step of applying heat lasts about 0.01 to 100 minutes.

According to another embodiment of the present invention, in each of the methods as defined above the pulsed electromagnetic field lasts about 0.01 to 100 minutes.

According to another embodiment of the present invention, in each of the methods as defined above the steps of applying heat and applying the pulsed electromagnetic therapy are simultaneous, sequential or separate.

According to another embodiment of the present invention, in each of the methods as defined above the method is repeated 1 to 100 times in each treatment.

According to another embodiment of the present invention, a typical protocol for the pulsed electromagnetic frequency generator (4) includes for example, and in a non limiting manner, a preset number of 1 microsecond period pulses with duty cycle of 50% and

a pause of up to 250 microsecond (in which the preset number of pulses correlates with energy to be supplied to skin under the treatment.)

According to another embodiment of the present invention, a typical protocol for the pulsed electromagnetic frequency generator (4) includes for example, and in a non limiting manner 10 pulses, of 1 microsecond period with 50% duty cycle and preset pause

of up to 512 microseconds (in which the pause correlates with energy to be supplied to skin under the treatment).

According to another embodiment of the present invention, a typical protocol for the pulsed electromagnetic frequency generator (4) includes for example, and in a non limiting manner, a repetition of the previous protocol, wherein the number of pulses administered is a multiplication of 10.

According to another embodiment of the present invention, in each of the methods as defined above the treatment is repeated more than once.

According to another embodiment of the present invention, each of the methods as defined above additionally comprising step of selecting the shape of said electromagnetic pulse is selected in a non-limiting manner from a group consisting of square wave, a sine wave, a triangular wave, sawtooth wave, ramp waves, spiked wave or any other mathematical combination.

Reference is now made to another embodiment of the present invention in which several applicators are being utilized for aesthetic purposes.

According to this embodiment, the first applicator comprises at least one RF electrode. The second applicator comprises an optical system to provide laser radiation to treat subcutaneous fat. The third applicator provides electrical current to the tissue so as to provide electro muscle stimulation.

Such device is an advanced medical aesthetic device that offers an effective solution for body aesthetic treatments through non-invasive lipolysis of the any desired body part (e.g., back, thighs abdomen and flanks), resulting in fat reduction. It employs diode laser applicators to reduce unwanted fat, combined with RF applicator and/or RF and pulsed electromagnetic filed (PEMF) applicator that combines Multi-Polar Radio Frequency and Pulsed Electro Magnetic Fields to reshape muscle, trigger lipolysis and tighten the skin, resulting in tighten skin and reduce circumference, resulting in a more contoured appearance of the treatment area.

The following provides disclosure as for the first and second applicators. The second applicator—optical system:

The optical system uses, according to one embodiment of the present invention, a 600μ×600μ square profile fiber and an aspheric lens to produce a square image of 60×60 mm after about 100 mm. The source is a multi-mode 600μ fiber with a numerical aperture of approx. 0.22.

Thus, the optical system comprises at least one laser source having a wavelength of about 800 nm-1200 nm; said optical system is adapted to provide homogeneous illumination to the skin surface to effect at least one target tissue at a depth of from about 1 mm to about 3 cm from said patient's skin's surface, at a power density in the range of 0.2 to 3 w/cm2 (most preferably, 1.4 W/cm²). more specifically, the wavelength is in the range of 1060 nm-1064 nm. The homogenous distribution is one with a substantially uniform light distribution under given conditions.

The 1064 nm diode laser applicators induce lipolysis in the desired body part (e.g., back, thighs abdomen and flanks), leading to destruction of fat cells, giving patients a solution to their stubborn fat deposits.

As a separate treatment, the first applicator (the RF or combined RF and PEMF) applicator allows a comprehensive body solution by tightening lax skin, leading to a more contoured appearance, as well as targeting cellulite on the abdomen, buttocks, and thighs for a smoother overall appearance.

The second applicator is a diode laser lipolysis treatment. The second applicator also includes a cooling mechanism and homogeneously energy distribution to avoid hot spots. Both the 1064 nm diode laser technology along with the first applicator (the RF and/or the combined RF with PEMF) are proven safe for all skin types.

The optical applicator (the second applicator) can be four diode laser applicators that can be activated individually and placed in various configurations on a belt (or any other wearable element such as a wearable accessory, accoutrement or garment; e.g., a strap, sleeve and any combination thereof). The treatment area of each diode laser applicator measures, e.g., 36 cm². With a wavelength of 1064 nm and the skin-contact cooling mechanism, the laser applicator energy is capable of effectively penetrating into the hypodermis to affect adipose tissue without harming the skin's surface. Over time, the body is able to naturally process and eliminate the disrupted fat cells through the lymphatic system.

Each applicator is equipped with user-friendly features to achieve lipolysis effectively, safely, and comfortably. The energy distribution across the laser applicator, contacting the patient's skin, is uniform for an even heating of target tissue, preventing hot spots in small areas and increasing the comfort.

Each individual laser applicator is equipped with four tissue-contact sensors to indicate appropriate contact with the patient's skin and ensure energy delivery only under these contact conditions, which ensures safety and the desired therapeutic effect. The top of each applicator features a red and green LED light to indicate proper and constant coupling of the applicator to the skin during the full course of the treatment.

There was no earlier mentioning of the sapphire crystal. You may add the mechanism of disabling laser delivery if no full contact occur

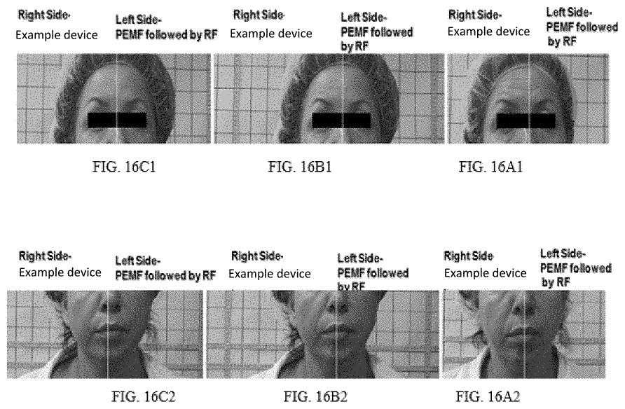

A unique belt design is also provided to allow a hands-free operation and ensures continuous contact of the applicators with the skin while assuring patient comfort throughout the treatment. Reference is now made to FIG. 17 illustrating the second applicator (in FIG. 2 optical applicators, 600, are illustrated) and the belt 601 to which said applicators are coupled so as to enable the hands-free operation. The coupling therebetween is enabled by simple mechanical connectors 602 (e.g., stretchers, strings etc.).

Each laser applicator is further enhanced with two internal temperature sensors that measure the water temperature coming in and out from the applicator. This is to ensure that the applicators, and the skin they are in contact with, are constantly cooled during the entire treatment.

As described above, the first applicator has a dual multi-polar electrode crown design, where the outer flat crown constantly controls volumetric heating, while the inner deep tetra-polar crown provides intermittent vacuum-assisted heating. According to one embodiment, vacuum is also provided. This, combined with the multi-polar technology, delivers a significant depth of impact of up to 4.5 cm for a greater effect on cellulite, while also affecting dermal connective tissue, likely via stimulation of fibroblast proliferation and collagen production.

According to one embodiment of the present invention, the first applicator is provided with an adjustable pulsed suction (vacuum) (e.g., a negative pressure means, a negative pressure source, negative pressure applicator, suction means) that facilitates a deep effect on tissue. The suction enhances the penetration and effects of RF energy by improving the contact surface between electrodes and tissue, thereby improving RF coupling and energy delivery. In addition to the coupling, it enables deeper impact due to the insertion of the tissue into the treatment chamber by the vacuum. Such pulsative suction also functions to increase blood flow and enhance lymphatic drainage.

According to one embodiment, Automatic Temperature Control (ATC), is also provided, which allows the operator to easily select a target temperature suitable for the specific patient and treatment area. This unique feature makes it easier to maintain a steady predetermined therapeutic temperature throughout the treatment, which enhances the consistency and predictability of results. The ATC may be implemented in the form of a programmable temperature regulator which may use pulse-width modulation and hysteresis control to maintain a temperature within a predetermined threshold of a selected temperature. For example, the ATC may employ a temperature sensor to determine a deviation between a sensed actual temperature and a preset (selected) temperature, and then activate or selectively turn on/off either heating and/or cooling if the sensed temperature is more than a given deviation from the selected temperature.

According to one embodiment of the present invention, either one of the applicators (the first, second or third) is also equipped with an integrated sensor that provides real-time thermal feedback, allowing for easy, immediate, and continuous monitoring of the skin's temperature profile.

The following table, table 3, lists the technical specification of the second application, the optical applicator:

Technical Specifications

| Input Power | | 100-240 VAC, less than 10 A, 50-60 Hz, single phase |
|---|---|---|
| Second applicator | Light Source | Diode Laser |
| Diode Laser Applications | Wavelength | [symbol] |
| | Max. Power | 50 watts |
| | Power Density | Up to 1.4 watts/cm² |
| | Spot Size | 36 cm² (6 cm × 6 cm) |
| First applicator | Max. RF Output Power | Up to 150 Watts |
| | Output RF Frequency | 1 Mhz |
| | Constant Magnetic Field | [symbol] |
| | Magnetic Pulse Frequency | 15 Hz |
| | Suction Levels | 4 |

| | |
|---|---|
| Screen Size | 12 in. |
| System Console Dimensions (W × D × H) | [symbol] |
| Weight | [symbol] |

[symbol]

indicates data missing or illegible when filed

The following will provide disclosure as for the third applicator. The third applicator adapted to provide electric muscle stimulation, EMS.

It is noted that the overall rational for the added applicator is improvement of entire skin/body shape and texture. While the first applicator (RF/PEMF) is intended for dermis, the second applicator is for deeper hypodermis (still affecting appearance of skin surface), and the 3rd applicator of EMS is for affecting the skin surface.

According to one embodiment of the present invention, at least one packet comprising a series of pulses. Each pulse is a symmetric biphasic pulse.

According to another embodiment, several packets are provided.

According to another embodiment, three different phases of EMS treatments are provided. According to this embodiment, the 3 phases:
a warm-up phase;
a contraction stage phase; and,
a relax phase.

According to one embodiment, the treatment time of the warm-up phase and/or the relax phase is at least 90 sec.

According to one embodiment, in the warm-up phase the applied frequency shifts and increases during that warm-up phase and in the relax phase the applied frequency decreases during the phase. According to one embodiment, the applied frequency, in the warm-up and the relax phases, is in the range of 1-100 Hz, most preferably, it is below 10 Hz.

The following table provides an exemplary embodiment of the applied frequency in the warm-up and the relax phases in the time frames thereof:

| Warm-up phase | Relax phase |
|---|---|
| Increase frequencies | Decrease frequencies |
| 3 Hz-30 sec | 7 Hz-30 sec |
| 4 Hz-30 sec | 6 Hz-30 sec |
| 5 Hz-30 sec | 5 Hz-30 sec |
| 6 Hz-30 sec | 4 Hz-30 sec |
| 7 Hz-30 sec | 3 Hz-30 sec |
| Total time: 2.5 min | Total time: 2.5 min |

According to one embodiment, the treatment time of the contraction phase is at least 7 minutes; more preferably, 20 minutes; most preferably, 25 minutes.

According to one embodiment, the contraction phase comprises at least one sub-treatment, each sub-treatment comprises 3 cycles, as listed in the following table:

| Cycle 1 | Cycle 2 | Cycle 3 |
|---|---|---|
| 50 Hz-180 sec | 150 Hz-120 sec | 5 Hz-120 sec |

According to one embodiment, the contraction phase comprises at least 3 reoccurring phases of the sub-treatment.

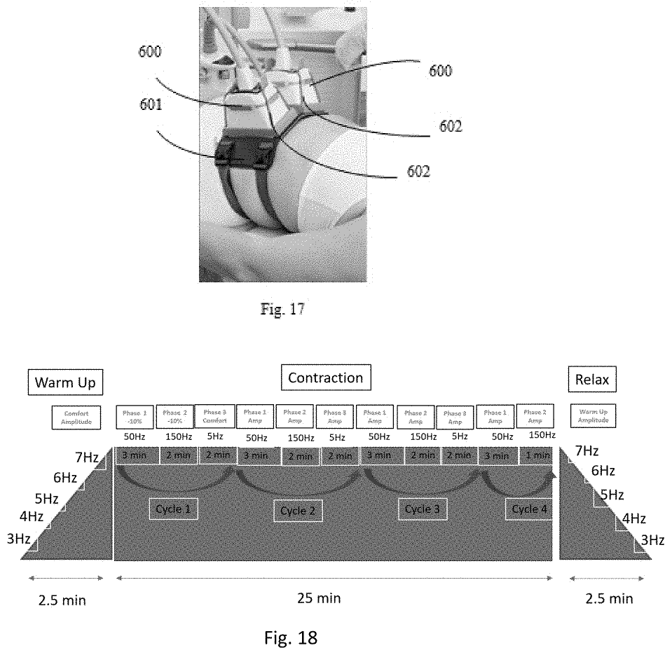

Reference is now made to FIG. 18, illustrating an exemplary EMS treatment. As seen in the figure, the EMS treatment comprises 3 phases:
a warm-up phase;
a contraction stage phase; and,
a relax phase.

Furthermore, as seen in FIG. 18, the warm-up and the relax phases takes 180 seconds and in which the frequencies increases (in the warm-up phase) and decreases (in the relax phase).

Also seen in FIG. 18, the contraction phase comprises several reoccurring phases of the sub-treatment, each comprises 3 cycles of 3 minutes of 50 Hz; 2 minutes of 150 Hz; and, 2 minutes of 5 Hz.

Thus, as disclosed above, according to one embodiment of the present invention, the shape of the electric waves is selected from the group consisting of a square wave, a sine wave, asymmetrical ladder, Trapezoid, symmetrical ladder, symmetrical bi phasic, a triangular wave, a sawtooth wave, a ramp wave, a spiked wave and any combination thereof.

According to another embodiment of the present invention, the frequency of the electric signal is in the range of about 1 Hz-250 Hz.

According to another embodiment of the present invention, the duration of the electric signal is in the range of about 400-5,000 microsecond.

According to another embodiment of the present invention, the frequency of the electric signal is in the range of about 40 Hz-2,500 Hz.

According to one embodiment of the present invention the shape of the signal is rectangular with waveform symmetric biphasic.

According to another embodiment of the present invention, the used frequency range is from about 1 to about 200 Hz; the pulse width is in the range of about 600 to about 1000 μS; the max intensity (output Voltage) is about 255 V.

According to another embodiment of the present invention, there are 2 electrodes being positioned vertically to one another.

According to another embodiment of the present invention, there are 2 electrodes being positioned horizontally to one another.

Thus, it is one object of the present invention to provide an integrated system for increasing skin rejuvenation of a region of a patient's skin and treating a subcutaneous fat region of said patient, said system comprising:
a. at least one applicator comprising:
at least one RF electrode adapted to be placed on said region of said patient's skin;
wherein none of said electrodes is configured to penetrate said skin; each of said electrodes is configured to apply heat up to a temperature T in a range of 25 degrees Celsius to 80 degrees Celsius;
b. at least one second applicator, in communication with an optical system, said optical system comprising:
at least one laser source having a wavelength of about 800 nm-1200 nm; said optical system is adapted to provide a homogeneous illumination to the skin surface to effect at least one target tissue at a depth of from about 1 mm to about 3 cm from said patient's skin's surface, at a power density in the range of 0.2 to 3 w/cm².

The subcutaneous fat may be fat associated with said region of the patient's skin, e.g., fat beneath the skin.

It is another object of the present invention to provide the system as defined above, wherein said at least one laser emitting energy is provided either in a continuous wave or a pulsed like manner.

It is another object of the present invention to provide the system as defined above, wherein said at least one laser emitting energy in the form of radiation having a wavelength of about 1060 nm-1064 nm.

It is another object of the present invention to provide the system as defined above, wherein said power density in the range of 0.8 to 1.4 w/cm².

It is another object of the present invention to provide the system as defined above, wherein said power density in the range of 0.8 to 2.25 w/cm².

It is another object of the present invention to provide the system as defined above, wherein said first applicator additionally comprising an electromagnetic field generator adapted to generate magnetic pulses.

It is another object of the present invention to provide the system as defined above, wherein said first applicator additionally comprising at least one coil.

It is another object of the present invention to provide the system as defined above, wherein at least one of said electrodes is encircled by at least one of said helical coils.

It is another object of the present invention to provide the system as defined above, wherein each of said electrodes encircled by at least one of said helical coils is provides RF energy and varied magnetic pulses to said region of a patient's skin.

It is another object of the present invention to provide the system as defined above, wherein said first applicator additionally comprising a control system.

It is another object of the present invention to provide the system as defined above, wherein said control system is adapted to control said electromagnetic field generator and application of RF by said heat by said electrodes (which is converted to heat due to impedance); and, all of said electrodes are adapted to simultaneously provide said electromagnetic pulses to said region of a patient's skin and to apply said heat to said region of a patient's skin.

It is another object of the present invention to provide the system as defined above, wherein said second applicator additionally comprising cooling means (e.g., a cooler, a chilling mechanism, a fan, an air source) adapted to cool said skin region.

It is another object of the present invention to provide the system as defined above, wherein said coil is a helical coil.

It is another object of the present invention to provide the system as defined above, wherein said heat applied to said region of a patient's skin is obtained by emitting RF radiation or via producing electrical current absorbed by subcutaneous tissue.

It is another object of the present invention to provide the system as defined above, wherein said system is adapted to provide a dynamic magnetic field such that said that varies with time.

It is another object of the present invention to provide the system as defined above, wherein each of said electromagnetic pulses has a shape selected from the group consisting of a square wave, a sine wave, a triangular wave, a sawtooth wave, a ramp wave, a spiked wave and any combination thereof.

It is another object of the present invention to provide the system as defined above, wherein said control system (6) monitors physical tissue parameters and changes said applied heat and said electromagnetic pulses accordingly.

It is another object of the present invention to provide the system as defined above, wherein a magnetic field intensity B of each pulse applied by said pulsed electromagnetic frequency generator (2) ranges between about 0 and about 3 Tesla.

It is another object of the present invention to provide the system as defined above, wherein a duration of each pulse applied by said system ranges between about 3 and about 1000 milliseconds.

It is another object of the present invention to provide the system as defined above, wherein a frequency F applied by the pulses of said system ranges between about 1 Hz and about 1M Hz.

It is another object of the present invention to provide the system as defined above, wherein an energy E applied by the pulses of said system ranges between about 1 and about 150 watts per pulse.

It is another object of the present invention to provide the system as defined above, wherein said applicator is in communication with said optical system by means of at least one optical fiber.

It is another object of the present invention to provide the system as defined above, wherein said at least one optical fiber is characterized by having a fiber core with a square cross section area.

It is another object of the present invention to provide the system as defined above, wherein said fiber core is characterized by dimensions of 200-600 micron.

It is another object of the present invention to provide the system as defined above, wherein the second applicator is characterized by at one dimension of the cross-section area of about 6 cm.

It is another object of the present invention to provide the system as defined above, additionally comprising a third applicator adapted to provide electric muscle stimulation.

It is another object of the present invention to provide the system as defined above, wherein the shape of the electric waves is selected from the group consisting of a square wave, a sine wave, asymmetrical ladder, Trapezoid, symmetrical ladder, symmetrical bi phasic, a triangular wave, a sawtooth wave, a ramp wave, a spiked wave and any combination thereof.

It is another object of the present invention to provide the system as defined above, wherein the frequency of the electric signal is in the range of about 1 Hz-250 Hz.

It is another object of the present invention to provide the system as defined above, wherein the duration of the electric signal is in the range of about 400-5,000 microsecond.

It is another object of the present invention to provide the system as defined above, wherein the frequency of the electric signal is in the range of about 40 Hz-2,500 Hz.

It is another object of the present invention to provide the system as defined above, wherein at least one of the following is being held true:
a. said first and second applicators are integrated into a single applicator;
b. said first and third applicators are integrated into a single applicator;
c. said third and second applicators are integrated into a single applicator;
d. said first, second and third applicators are integrated into a single applicator.

It is another object of the present invention to provide the system as defined above, wherein said first and second applicators are integrated into a single applicator.

It is another object of the present invention to provide a method of increasing skin rejuvenation of a region of a patient's skin and treating a subcutaneous fat region of said patient, comprising step of:
a. obtaining the system of any of the defined above,
b. applying either simultaneously or in batch like manner (a) heat to a subcutaneous tissue within said region up to temperature T; (c) radiation in the form of a laser radiation having a wavelength of about 800 nm-1200 nm; or any combination thereof.

It is another object of the present invention to provide the method as defined above, wherein said laser is characterized by having power density in the range of 0.2 to 3 w/cm².

It is another object of the present invention to provide the method as defined above, wherein said laser radiation is provided either in a continuous wave or a pulsed like manner.

It is another object of the present invention to provide the method as defined above, wherein said at least one laser emitting energy in the form of radiation having a wavelength of about 1060 nm-1064 nm.

It is another object of the present invention to provide the method as defined above, wherein said power density in the range of 0.8 to 1.4 w/cm².

It is another object of the present invention to provide the method as defined above, wherein said power density in the range of 0.8 to 2.25 w/cm².

It is another object of the present invention to provide the method as defined above, wherein said second applicator additionally comprising cooling means adapted to cool said skin region.

It is another object of the present invention to provide the method as defined above, wherein said first applicator additionally comprising an electromagnetic field generator adapted to generate magnetic pulses.

It is another object of the present invention to provide the method as defined above, wherein said first applicator additionally comprising at least one coil.

It is another object of the present invention to provide the method as defined above, wherein at least one of said electrodes is encircled by at least one of said helical coils.

It is another object of the present invention to provide the method as defined above, wherein each of said electrodes encircled by at least one of said helical coils is provides RF energy and varied magnetic pulses to said region of a patient's skin.

It is another object of the present invention to provide the method as defined above, wherein said first applicator additionally comprising a control system.

It is another object of the present invention to provide the method as defined above, wherein said control system is adapted to control said electromagnetic field generator and application of said heat by said electrodes; and, all of said electrodes are adapted to simultaneously provide said electromagnetic pulses to said region of a patient's skin and to apply said heat (by means of RF energy) to said region of a patient's skin.

It is another object of the present invention to provide the method as defined above, additionally comprising a step of monitoring and/or controlling said steps of applying heat to a subcutaneous tissue within said region and/or said step of applying pulsed electromagnetic therapy to said region.

It is another object of the present invention to provide the method as defined above, additionally comprising a step of applying a time varying dynamic magnetic field onto said region.

It is another object of the present invention to provide the method as defined above, additionally comprising step of a. storing in a communicable database predetermined parameters defining (i) safe treatment parameters and (ii) unsafe treatment parameters; said parameters are selected from a group consisting of time t of said treatment, temperature T of said tissue, ratio t/T, Frequency F, power P, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof; b. sensing electromagnetic radiation and heat radiation parameters selected from a group consisting of time t of said treatment, temperature T of said tissue, ratio t/T, Frequency F, power P, Intensity I of said ultrasound irradiation, energy E applied by the pulses of said pulsed electromagnetic frequency generator, depth D of said treated tissue, magnetic field intensity B, tissue impedance, specific absorption rate (SAR), treatment depth, superficial muscle contractions or a combination thereof; c. allowing said electromagnetic radiation and said heat radiation if parameters within said safe treatment parameters and to stop the electromagnetic radiation if the radiation parameters are in said unsafe treatment parameters.

It is another object of the present invention to provide the method as defined above, additionally comprising a step of selecting the shape of said electromagnetic pulse is selected in a non-limiting manner from a group consisting of square wave, a sine wave, a triangular wave, sawtooth wave, ramp waves, spiked wave or any other mathematical combination.

It is another object of the present invention to provide the method as defined above, additionally comprising a step of applying a series of electromagnetic pulses, said series of electromagnetic pulses comprising recurring applications of a pattern comprising a preset number of square pulses of duration of about 1 microsecond with a duty cycle of 50% followed by a pause of not more than about 250 microseconds.

It is another object of the present invention to provide the method as defined above, additionally comprising a step of selecting said deep tissue diathermy device (4) from any device emitting RF radiation or any means adapted for producing electrical current absorbed by subcutaneous tissue.

It is another object of the present invention to provide the method as defined above, additionally comprising a step of selecting said deep tissue diathermy device (4) comprises at least two electrodes adapted to be placed on said region of said patient's skin.

It is another object of the present invention to provide the method as defined above, wherein said step of (a) obtaining (i) pulsed electromagnetic frequency generator; and (ii) a deep tissue diathermy device, comprising steps of: a. obtaining at least two electrodes adapted to be placed on said region of said patient's skin; an electromagnetic field generator adapted to generate magnetic pulses; and, at least one coil; wherein at least one of said electrodes is encircled by at least one of said helical coils.

It is another object of the present invention to provide the method as defined above, wherein said step of applying heat to a subcutaneous tissue additionally comprising step of a. obtaining at least one electrical output device adapted to generate either RF electromagnetic energy or electrical current; b. electrically coupling at least two electrodes to said electrical output device; c. placing said at least two electrodes on said skin region; and, d. simultaneously applying via all said electrodes said RF energy or said electrical current to said skin region.

It is another object of the present invention to provide the method as defined above, additionally comprising a step of selecting at least one parameter from a group consisting of (a) the duration of each pulse applied by said pulsed electromagnetic frequency generator (2) to be greater than about 3 and lower than about 1000 milliseconds; (b) the magnetic field intensity B of each pulse applied by said step of applying pulsed electromagnetic therapy to said region to be higher than about 0 and lower than about max magnetic field used in MRI devices (i.e., 3 Tesla); (c) the frequency F applied by the pulses applied by said step of applying pulsed electromagnetic therapy to said region to be higher than about 1 and lower than about 1M Hz; (d) the energy E applied by said step of applying pulsed electromagnetic therapy to said region to be greater than about 1 and lower than about 150 watts per pulse.

It is another object of the present invention to provide the method as defined above, wherein step of applying heat is applied for about 0.01 to 60 minutes.

It is another object of the present invention to provide the method as defined above, wherein the RF (and the heat thereof) and the pulsed electromagnetic therapy are applied simultaneously, sequentially or separately.

It is another object of the present invention to provide the method as defined above, wherein said step of applying heat is performed by devices selected from a group consisting of: ultrasonic diathermy, an optical device, electromagnetic induction, sound waves emitting instrument, direct heat applying instrument, or from any other means of heating subcutaneous tissue to temperature T.

It is another object of the present invention to provide the method as defined above, wherein said applicator is in communication with said optical system by means of at least one optical fiber.

It is another object of the present invention to provide the method as defined above, wherein said at least one optical fiber is characterized by having a fiber core with a square cross section area.

It is another object of the present invention to provide the method as defined above, wherein said fiber core is characterized by dimensions of 200-600 micron.

It is another object of the present invention to provide the method as defined above, wherein the second applicator is characterized by at one dimension of the cross section area of about 6 cm.

It is another object of the present invention to provide the method as defined above, additionally comprising a third applicator adapted to provide electric muscle stimulation.

It is another object of the present invention to provide the method as defined above, wherein the shape of the electric waves is selected from the group consisting of a square wave, a sine wave, asymmetrical ladder, Trapezoid, symmetrical ladder, symmetrical bi phasic, a triangular wave, a sawtooth wave, a ramp wave, a spiked wave and any combination thereof.

It is another object of the present invention to provide the method as defined above, wherein the frequency of the electric signal is in the range of about 1 Hz-250 Hz.

It is another object of the present invention to provide the method as defined above, wherein the pulse length of the electric signal is in the range of about 400-5,000 microsecond.

It is another object of the present invention to provide the method as defined above, wherein the frequency of the electric signal is in the range of about 40 Hz-2,500 Hz.

It is another object of the present invention to provide the method as defined above, wherein at least one of the following is being held true:
a. said first and second applicators are integrated into a single applicator;
b. said first and third applicators are integrated into a single applicator;
c. said third and second applicators are integrated into a single applicator;
d. said first, second and third applicators are integrated into a single applicator.

It is another object of the present invention to provide the method as defined above, wherein said first and second applicators are integrated into a single applicator.

It should also be appreciated that the above described description of methods and apparatus are to be interpreted as including apparatus for carrying out the methods, and methods of using the apparatus of any type as well known to a person or ordinary skill, and which need not be described in detail herein for enabling a person of ordinary skill to practice the invention.

For the main embodiments of the invention, the particular selection of type and model is not critical, though where specifically identified, this may be relevant. The present invention has been described using detailed descriptions of embodiments thereof that are provided by way of example and are not intended to limit the scope of the invention. No limitation, in general, or by way of words such as “may”, “should”, “preferably”, “must”, or other term denoting a degree of importance or motivation, should be considered as a limitation on the scope of the claims or their equivalents unless expressly present in such claim as a literal limitation on its scope. It should be understood that features and steps described with respect to one embodiment may be used with other embodiments and that not all embodiments of the invention have all of the features and/or steps shown in a particular figure or described with respect to one of the embodiments. That is, the disclosure should be considered complete from combinatorial point of view, with each embodiment of each element considered disclosed in conjunction with each other embodiment of each element (and indeed in various combinations of compatible implementations of variations in the same element). Variations of embodiments described will occur to persons of the art. Furthermore, the terms “comprise,” “include,” “have” and their conjugates, shall mean, when used in the claims, “including but not necessarily limited to.” Each element present in the claims in the singular shall mean one or more element as claimed, and when an option is provided for one or more of a group, it shall be interpreted to mean that the claim requires only one member selected from the various options, and shall not require one of each option. The abstract shall not be interpreted as limiting on the scope of the application or claims.

It is noted that some of the above described embodiments may describe the best mode contemplated by the inventors and therefore may include structure, acts or details of structures and acts that may not be essential to the invention and which are described as examples. Structure and acts described herein are replaceable by equivalents performing the same function, even if the structure or acts are different, as known in the art. Therefore, the scope of the invention is limited only by the elements and limitations as used in the claims.

### EXAMPLES

Examples are given in order to prove the embodiments claimed in the present invention. The example, which is a clinical test, describes the manner and process of the present invention and set forth the best mode contemplated by the inventors for carrying out the invention, but are not to be construed as limiting the invention.

### Example 1

A clinical test was performed to demonstrate the synergic effect of the combined PEMF and the deep tissue diathermy device.

The aim of the example is to evaluate the synergetic effect of the combined technology and compare it to each technology individually.

A multi polar magnetic pulsed synthesizer which simultaneously emits RF and magnetic pulses in varying phases that homogeneously cause supra normal temperatures over the treated area and penetrates the dermis and hypodermis was used.

Method

The test included 40 women at the age of 42-61 years.

They were divided to 4 groups; 1 study group and 3 control groups, each group included 10 clients. All participants were selected very punctiliously according to predefined criterions which included loosen skin in the forehead, eyes and neck area.

All groups were treated for skin tightening of the face (forehead & eyes) and neck. In all groups the right side of the face and neck was treated only by the use of combined technology and the left side of the face and neck was treated according the following:

Study group: the combined technology as well.

First control group: RF technology.

Second control group: PMF (Pulsed Magnetic Field) technology.

Third control group: PMF technology and 2 hours later RF technology.

Sessions were conducted once a week for a period of 8 weeks.

Each session lasted 40 minutes (20 minutes each side) except of the third control group which lasted 60 minutes (right side 20 minutes and left side 40 minutes because we used 2 different technologies).

Clinical results were collected in two paths; objective and subjective.

### Objective Method

Objective method was conducted by taking photographs in order to assess the changes in the tightness of the skin induced by the treatments.

The pictures were taken before and after treatments by the same operator under the same conditions. The pictures were taken while the participant sat in front of a chart with vertical and horizontal lines with the camera placed at the same height distance and with the same lightening conditions.

### Subjective Method

Subjective method was conducted by clients' self report.

The subjective method was conducted by questions that each client had to fill after every treatment, referring the immediate results they had noticed on each treated side, the notice of accumulative effect, the satisfaction of the patients from the results, sensation of the treatments etc.

The patients received satisfaction questionnaire which included yes/no questions and questions to be grade on 1 to 5 satisfaction scales (1—represents “Not At All” and 5 represents “Very Much”).

Additionally there were open questions adapted to enable the patients the ability to express any kind of feeling following the treatments.

The following questionnaire was handed to the patients after each treatment (except of specific questions):

| Question | Score | | | | | Full Answer |
|---|---|---|---|---|---|---|
| Do you see any visual changes on the | Not at | | | | Very | |
| right side that was treated? | all | | | | much | |
| Please describe in the “Full Answer” | 1 | 2 | 3 | 4 | 5 | |
| column. | | | | | | |
| Are you satisfied from the visual | Not at | | | | Very | |
| changes on the right | all | | | | much | |
| side that was treated? | 1 | 2 | 3 | 4 | 5 | |
| Is there any specific feeling you feel on | Yes/No | | | | | |
| the right side that was treated (tightness, | | | | | | |
| lifting, stretched, fullness, swelled, | | | | | | |
| loosen, ache, scratching)? | | | | | | |
| Please describe in the “Full Answer” | | | | | | |
| column. | | | | | | |
| Are you satisfied from the feeling | Not at | | | | Very | |
| you feel on the right | all | | | | much | |
| side that was treated? | 1 | 2 | 3 | 4 | 5 | |
| Do you see any visual changes on the | Not at | | | | Very | |
| left side that was treated? | all | | | | much | |
| Please describe in the “Full Answer” | 1 | 2 | 3 | 4 | 5 | |
| column. | | | | | | |
| Are you satisfied from the visual | Not at | | | | Very | |
| changes on the left | all | | | | much | |
| side that was treated? | 1 | 2 | 3 | 4 | 5 | |
| Is there any specific feeling you feel on | Yes/No | | | | | |
| the left side that was treated (tightness, | | | | | | |
| lifting, stretched, fullness, swelled, | | | | | | |
| loosen, ache, scratching)? | | | | | | |
| Please describe in the “Full Answer” | | | | | | |
| column. | | | | | | |
| Are you satisfied from the feeling | Not at | | | | Very | |
| you feel on the left | all | | | | much | |
| side that was treated? | 1 | 2 | 3 | 4 | 5 | |

| | | |
|---|---|---|
| Answer only | How long did the visual results last on | |
| from the | the right side that was treated (Please | |
| second | describe in the “Full Answer” column)? | |
| treatment | | |
| Answer only | How long did the visual results last on | |
| from the | the left side that was treated (Please | |
| second | describe in the “Full Answer” column)? | |
| treatment | | |
| Answer only | Do you feel that the results on the left | Yes/No |
| after the 5^{th} | side that was treated were accumulated | |
| treatment | from the first treatment? | |
| Answer only | Do you feel that the results on the right | Yes/No |
| after the 5^{th} | side that was treated were accumulated | |
| treatment | from the first treatment? | |
| Answer | Do you feel that the results on the left | Yes/No |
| after the 8^{th} | side that was treated were accumulated | |
| treatment | from the first treatment? | |
| Answer | Do you feel that the results on the right | Yes/No |
| after the 8^{th} | side that was treated were accumulated | |
| treatment | from the first treatment? | |

General
comments

### Results

### Study Group

All patients have shown immediate and highly noticeable results on both sides, after the first treatment; the skin tightness of the face and neck increased, it was smoother and with less wrinkles.

Further, after 5 treatments it was noticeable that the results were accumulated (based on comparison between pictures before the first treatment and pictures before the 5^{th} treatment). After 8 treatments the skin tightness increased significantly. All clients indicated great satisfaction from the immediate visual results (tightness & stretched) as well as the long term results.

The average score of visual results and satisfaction on the right treated side was 4.66 (on 1 to 5 scale) and on the left treated side the average score was 4.8. 100% of the clients reported that the results on both sides remained all week along the sessions and were accumulated.

They report feelings of tightness, firm and highly comfort during the treatment in terms of the sensation (“Feels like hot stones massage”) and expressed satisfaction from the sort time of the treatment.

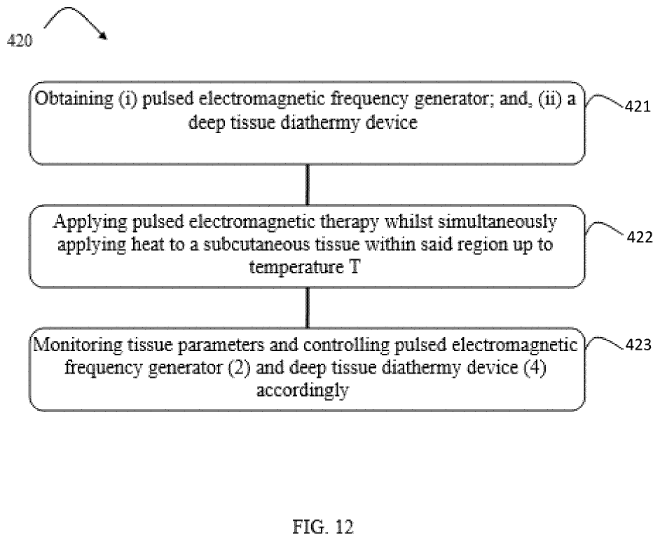

Reference is now made to FIG. 13A1 which are pictures of one patient out of the study group treated with the device of the present invention. The pictures were taken before the treatment (see FIG. 13A1 for the forehead and FIG. 13A2 for the neck), after the first treatment (see FIG. 13B1 for the forehead and FIG. 13B2 for the neck) and after the 8^{th} treatment (see FIG. 13C1 for the forehead and FIG. 13C2 for the neck).

### First Control Group

All patients have shown immediate results on both sides, after the first treatment. However the results were more significant in terms of tightening and wrinkles fading on the right side compare to the left side.

The average score of visual results and satisfaction on the right treated side was 4.93 compare to the left treated side which was 4.8.

Following 5 treatments it was noticeable that the results were accumulated significantly on the right side in comparison to the results on the left side which lasted only for few days. 90% of the clients reported that the results on the right side remained all week along the first 5 treatments and were accumulated compare to 40% of the clients that reported maintenance of the results on the left side.

After 8 treatments the skin tightness increased even more on the right side and 100% of the clients reported accumulative results compare to the results of the left side which remained almost the same and only 50% of the clients reported maintenance of the results.

Both clients indicated higher satisfaction from the immediate and long term results on the right side compare to the left side. No difference between both treated sides in terms of comfort was expressed.

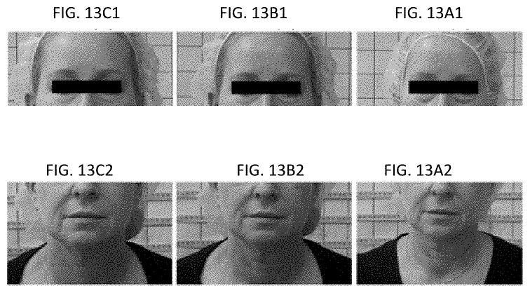

Reference is now made to FIGS. 14A1-14C2 which are pictures of one patient out of the first control group treated with the device of the present invention on the right side and RF on the left side. The pictures were taken before the treatment (see FIG. 14A1 for the forehead and FIG. 14A2 for the neck), after the first treatment (see FIG. 14B1 for the forehead and FIG. 14B2 for the neck) and after the 8^{th} treatment (see FIG. 14AC1 for the forehead and FIG. 14C2 for the neck).

### Second Control Group

All patients have shown immediate and very noticeable results (skin tightening and wrinkles fading) on the right side after the first treatment and the results have improved after the 5^{th} and the 8^{th} treatment.

The average score of visual results and satisfaction on the right treated side was 5.00 compare to the left treated side which was only 1.9.

It should be pointed out that on the left side they didn't show any results in terms of skin tightness along the treatments, although after the 4^{th} treatment the skin showed some improvement (it looked more glowing and nourished).

All clients indicated high satisfaction from the immediate and long term results on the right side. 100% of the clients reported that the results on the right side remained all week along the first 5 treatments and were accumulated (and up to the 8^{th} treatment) compare to 10% of the clients that reported maintenance of the results on the left side.

As for the left side, they reported after the 5^{th} treatment satisfaction from the improved skin's condition and look, although they expressed some disappointment from not having results in terms of skin tightening. They reported high comfort in terms of the treatment sensation during the treatment of both sides.

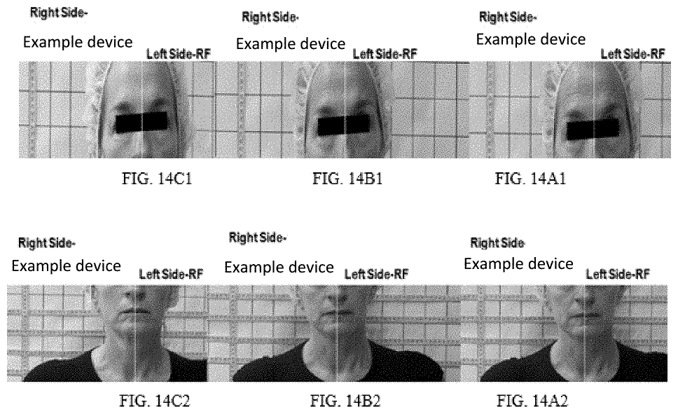

Reference is now made to FIGS. 15A1-15C2 which are pictures of one patient out of the second control group treated with the device of the present invention on the right side and PEW′ on the left side. The pictures were taken before the treatment (see FIG. 15A1 for the forehead and FIG. 15A2 for the neck), after the first treatment (see FIG. 15B1 for the forehead and FIG. 15B2 for the neck) and after the 8^{th} treatment (see FIG. 15C1 for the forehead and FIG. 15C2 for the neck).

### Third Control Group

All patients have shown on the right side very noticeable immediate and accumulative results (skin tightening and wrinkles fading).

The satisfaction was very high.

On the left side visual results are seen; the immediate results were similar to the right side, however the accumulative results were less significant and noticeable compare with the left side.

The average score of visual results and satisfaction on the right treated side was 4.83 compare to the left treated side which was only 2.36.

90% of the clients reported that the results on the right side remained all week along the first 5 treatments (and up to the 8^{th} treatment) and were accumulated.

Only 30% of the clients reported maintenance of the results on the left side. In term of satisfaction the clients expressed inconvenient due to the long duration of the treatment.

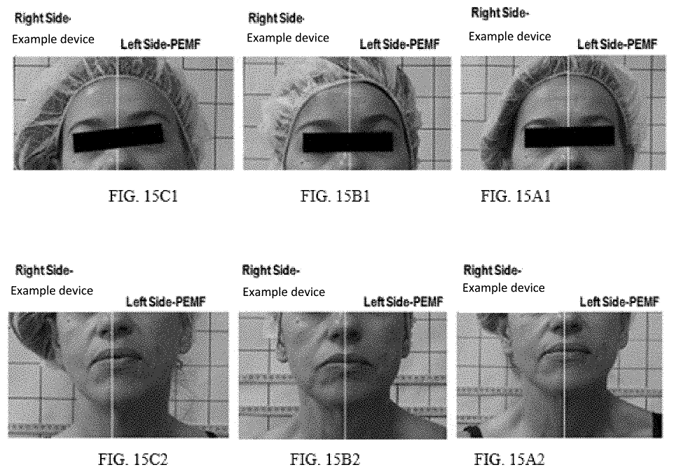

Reference is now made to FIGS. 16A1-16C2 which are pictures of one patient out of the third control group treated with the device of the present invention on the right side. The left side was treated with PEMF followed by RF. The pictures were taken before the treatment (see FIG. 16A1 for the forehead and FIG. 16A2 for the neck), after the first treatment (see FIG. 16B1 for the forehead and FIG. 16B2 for the neck) and after the 8^{th} treatment (see FIG. 16C1 for the forehead and FIG. 16C2 for the neck).

The following tables (tables 3 and 4) summaries the results:

TABLE 4
average score (1 to 5 scale) of all participant in each group following all the treatments:

| Question | Study group | Control 1 | Control 2 | Control 3 |
|---|---|---|---|---|
| Do you see any visual changes on | 4.7 | 4.9 | 5 | 4.9 |
| the right side that was treated? | | | | |
| Are you satisfied from the visual | 4.7 | 5 | 5 | 4.8 |
| changes on the right side that was | | | | |
| treated? | | | | |
| Are you satisfied from the feeling | 4.6 | 4.9 | 5 | 4.8 |
| you feel on the right side that was | | | | |
| treated? | | | | |
| Do you see any visual changes on | 4.9 | 2.5 | 1.9 | 2.4 |
| the left side that was treated? | | | | |
| Are you satisfied from the visual | 4.8 | 2.6 | 2 | 2.3 |
| changes on the left side that was | | | | |
| treated? | | | | |
| Are you satisfied from the feeling | 4.7 | 2.4 | 1.8 | 2.4 |
| you feel on the left side that was | | | | |
| treated? | | | | |

TABLE 5
results of “Yes/No” Questions of all the patients in each group following all the treatments

| | Study | | | | | | | |
|---|---|---|---|---|---|---|---|---|
| | group | | Control 1 | | Control 2 | | Control 3 | |
| Question | Yes | No | Yes | No | Yes | No | Yes | No |
| Is there any specific feeling you | 80% | 20% | 90% | 10% | 100% | 0% | 80% | 20% |
| feel on the right side that was | | | | | | | | |
| treated? (tightness, lifting | | | | | | | | |
| stretched, fullness, swelled, loosen, | | | | | | | | |
| ache, scratching)? | | | | | | | | |
| Is there any specific feeling you | 90% | 10% | 60% | 40% | 40% | 60% | 50% | 50% |
| feel on the left side that was | | | | | | | | |
| treated? (tightness, lifting | | | | | | | | |
| stretched, fullness, swelled, loosen, | | | | | | | | |
| ache, scratching)? | | | | | | | | |
| Answer only after the 5th | | | | | | | | |
| treatment: | | | | | | | | |
| Do you feel that the results on the | 100% | 0% | 40% | 60% | 10% | 90% | 30% | 70% |
| left side that was treated were | | | | | | | | |
| accumulated from the first | | | | | | | | |
| treatment? | | | | | | | | |
| Answer only after the 5th | | | | | | | | |
| treatment: | | | | | | | | |
| Do you feel that the results on the | 100% | 0% | 90% | 10% | 100% | 0% | 90% | 10% |
| right side that was treated were | | | | | | | | |
| accumulated from the first | | | | | | | | |
| treatment? | | | | | | | | |
| Answer only after the 8th | | | | | | | | |
| treatment: | | | | | | | | |
| Do you feel that the results on the | 100% | 0% | 50% | 50% | 20% | 80% | 30% | 70% |
| left side that was treated were | | | | | | | | |
| accumulated from the first | | | | | | | | |
| treatment? | | | | | | | | |
| Answer only after the 8th | | | | | | | | |
| treatment: | | | | | | | | |
| Do you feel that the results on the | 100% | 0% | 100% | 0% | 100% | 0% | 100% | 0% |
| right side that was treated were | | | | | | | | |
| accumulated from the first | | | | | | | | |
| treatment? | | | | | | | | |

### CONCLUSIONS

The synergetic effect of the device of the present invention clearly shows objectively and subjectively superior results when compare to the treatments in which only RF, or only PEMF were used.

It has shown that clients who were treated on the left side with PEMF technology had a much clearer difference between both sides. This is probably since they have not seen any tightness effect on the left side.

In addition it was shown that clients that were treated with the device of the present invention on both sides felt significant changes along the treatment. Yet more, it was harder for them to see the difference between both sides since both sides were treated with the device of the present invention and both sides had improved in the same way. 80%-100% out of all 40 participants answered “Yes” regarding questions concerning the tightness of the skin and the accumulative results on the right treated area. Only 10%-50% that were treated on the left side with other technology answered “Yes” regarding questions concerning the tightness of the skin and the accumulative results.

The results with the combined technology (the device provided by the present invention) were immediate and they maintained and improved from one treatment to the other. Immediate skin tightening has been seen due to the change of collagen fibers formation (they become shorter and thicker and as a result harder) induced by thermal technique of the RF.

Long lasting results has been seen due to the increase of new collagen fibers synthesis by using thermal (RF) and non-thermal (PEMF) technologies induced by the device of the present invention enables the formation change of greater amount of collagen fibers and as a results created physiological buttress that enabled better structural support of the skin.

The assets of the device of the present invention to the medical field are:
a. Synergistic effect that stimulates dermal fibroblasts which produce new collagen, elastic and reticular fibers by using different mechanisms (Heating & non heating);
b. Changing the form of a greater amount of collagen fiber by making them shorter and thicker; and,
c. Angiogenesis—increasing the formation of new small blood vessels.

1. An integrated system for increasing skin rejuvenation of a region of a patient's skin and treating subcutaneous fat associated with the region, said system comprising:
a. at least one first applicator comprising:
one or more RF electrodes adapted to be placed on said region of said patient's skin;
wherein none of said RF electrodes is configured to penetrate said skin; and at least one of said RF electrodes is configured to apply RF energy to said region of the patient's skin to heat up to a temperature T in a range of 25 degrees Celsius to 80 degrees Celsius;
b. at least one second applicator, in communication with an optical system, said optical system comprising:
at least one laser source having a wavelength of about 800 nm-1200 nm,
said optical system being adapted to provide homogeneous illumination to the skin surface to effect at least one target tissue at a depth of from about 1 mm to about 3 cm from said patient's skin's surface, at a power density in the range of 0.2 to 3 w/cm².

2. The system according to claim 1, wherein said at least one laser source is configured to emit laser energy either in a continuous wave or a pulsed like manner.

3. The system according to claim 1, wherein said at least one laser source is configured to emit energy in the form of radiation having a wavelength of about 1060 nm-1064 nm.

4. The system according to claim 1, wherein said power density in the range of 0.8 to 1.4 w/cm².

5. The system according to claim 1, wherein said power density in the range of 0.8 to 2.25 w/cm².

6. The system according to claim 1, wherein said at least one first applicator additionally comprises an electromagnetic field generator adapted to generate magnetic pulses.

7. The system according to claim 6, wherein said at least one first applicator additionally comprising at least one coil.

8. The system according to claim 7, wherein at least one of said RF electrodes is encircled by said at least one coil.

9. The system according to claim 8, wherein each of said electrodes encircled by at least one of said coils is configured to provide RF energy and varied magnetic pulses to said region of a patient's skin.

10. The system according to claim 1, wherein system additionally comprises at least one control system.

11. The system according to claim 10, wherein said control system is adapted to control at least one parameter selected from a group consisting of (a) temperature applied to said skin by said at least one RF electrode; (b) contact of either one of said first at least one and/or said at least one second applicator with said skin; (c) the frequency of said magnetic pulses; (d) the magnetic field's strength or flux; (e) the duration of said magnetic pulses; (f) the power applied by said at least one RF electrode; (g) the power density of said second applicator; (h) the wavelength of said at least one laser source; (i) emission of RF energy by said at least one RF electrode, simultaneously with emission of said magnetic pulses to said region of a patient's skin; and any combination thereof.

12. The system according to claim 1, wherein said second at least one applicator additionally comprising a cooler adapted to cool said skin region.

13. The system according to claim 6, wherein the shape of said coil is selected from a group consisting of a helical coil, a spiral coil, a flat coil, a planar coil, a circular coil, a helical coil with a pitch and any combination thereof.

14. The system according to claim 6, wherein said magnetic field is a time varying magnetic field.

15. The system according to claim 14, wherein the shape of at least one pulse applied by said dynamic magnetic field is selected from the group consisting of a square wave, a sine wave, a triangular wave, a sawtooth wave, a ramp wave, a spiked wave and any combination thereof.

16. The system according to claim 14, wherein a magnetic flux ranges between about 15 gauss and about 3 Tesla.

17. The system according to claim 14, wherein a duration of each pulse of said magnetic pulses ranges between about 0.2 and about 1000 milliseconds.

18. The system according to claim 14, wherein a frequency of said magnetic pulses ranges between about 1 Hz and about 1M Hz.

19. The system according to claim 1, wherein power of said at least one RF electrode ranges between about 1 and about 250 watts and frequency of said pulses ranges between about 1 MHz-40 MHz.

20. The system according to claim 1, wherein said second applicator is in communication with said optical system by means of at least one optical fiber.

21. The system according to claim 20, wherein said at least one optical fiber is characterized by having a fiber core having a cross sectional area selected from a group consisting of a square cross section area, a rectangular cross section area, an elliptical cross section area, a circular cross section area, a triangular cross section area, pentagonal cross section area, a hexagonal cross section area, a heptagonal cross section area, an octagonal cross section area, a nonagonal cross-section area, a decagonal cross section area, and any combination thereof.

22. The system according to claim 21, wherein said fiber core is characterized by dimensions of 100-1000 micron.

23. The system according to claim 1, additionally comprising at least one third applicator adapted to provide electrical signal so as to provide electrical stimulation.

24. The system according to claim 23, wherein the shape of the electric signal is selected from the group consisting of a square wave, a sine wave, an asymmetrical ladder, a trapezoid, a symmetrical ladder, symmetrical bi phasic, asymmetrical bi phasic, a triangular wave, a sawtooth wave, a ramp wave, a spiked wave and any combination thereof.

25. The system according to claim 23, wherein said electric signal is adapted to provide electrical stimulation stimulate at least one selected from a group consisting of muscles, tissue, nerve and any combination thereof.

26. The system according to claim 23, wherein said electric signal is adapted to provide transcutaneous electrical nerve stimulation.

27. The system according to claim 23, wherein the frequency of the electric signal is in the range of about 1 Hz-2500 kHz.

28. The system according to claim 23, wherein the pulse length of the electric signal is in the range of about 100-5,000 microsecond.

29. The system according to claim 23, wherein at least one of the following is being held true:
a. said at least one first and at least one second applicators are integrated into a single applicator;
b. said at least one first and at least one third applicators are integrated into a single applicator;
c. said at least one third and at least one second applicators are integrated into a single applicator;
d. said at least one first, at least one second and at least one third applicators are integrated into a single applicator.

30. The system according to claim 23, wherein said electrical stimulation comprises at least three phases including (a) a warm-up phase; (b) a contraction phase; and, (c) a relax phase.

31. The system according to claim 30, wherein in said warm-up phase, the frequency of the applied the electric signal increases.

32. The system according to claim 30, wherein in said relax phase, the frequency of the applied the electric signal decreases.

33. The system according to claim 30, wherein said contraction phase comprises at least one cycle of at least 3 sub-treatments: (a) a first frequency, F1, of the applied the electric signal for a first predetermined period of time, T1; (b) a second frequency, F2, of the applied the electric signal for a second predetermined period of time, T2; (c) a third frequency, F3, of the applied the electric signal for a third predetermined period of time, T3.

34. The system according to claim 33, wherein said F1 is in the range of 0 to 100 Hz; F2 is in the range of 50 to 1000 Hz; F3 is in the range of 0 to 50 Hz; T1, T2 and T3 are in the range of 0-10 minutes.

35. The system according to claim 1, wherein said first and second at least one applicators are integrated into a single applicator.

36. The system according to claim 29, wherein said first at least one applicator additionally comprising an electromagnetic field generator adapted to generate magnetic pulses.

37. The system according to claim 1, comprising at least one selected from a group consisting of a plurality of first applicators, a plurality of second applicators, a plurality of third applicators and any combination thereof.

38. The system according to claim 37, wherein each of the applicators are activated individually.

39. The system according to claim 1, additionally comprising at least one wearable accessory adapted to be worn be the treated patient, adapted to fixate the position of at least one of said applicators on said patient's skin.

40. The system according to claim 39, wherein said wearable accessory is selected from a group consisting of a belt, a strap, a sleeve and any combination thereof.

41. The system according to claim 39, wherein said wearable accessory is reversibly coupled to at least one selected from a group consisting of a first applicator, a second applicator, a third applicator and any combination thereof.

42. The system according to claim 41, wherein said wearable accessory is adapted to provide a hands-free operation of each of said applicators on said patient's skin.

43. The system according to claim 1, wherein at least one selected from a group consisting of a first applicator, a second applicator, a third applicator and any combination thereof additionally comprises at least one tissue-contact sensor adapted to indicate contact with said patient's skin.

44. The system according to claim 43, wherein said tissue-contact sensor is an LED light adapted to indicate proper and constant coupling of the applicator to said patient's skin during at least part of the treatment.

45. The system according to claim 1, wherein at least one selected from a group consisting of first applicator, second applicator, third applicator and any combination thereof additionally comprising at least one temperature sensor.

46. The system according to claim 1, wherein at least one selected from a group consisting of first applicator, second applicator, third applicator and any combination thereof additionally comprises a negative pressure source.

47. The system according to claim 46, wherein said negative pressure applied to said skin is adjustable pulsed suction adapted to facilitate a deeper effect on said patient's skin.

48. The system according to claim 1, additionally comprising an Automatic Temperature Control (ATC), adapted to set at least one target temperature required.

49. The system according to claim 1, additionally comprising at least one sensor adapted to provide a real-time thermal feedback from said tissue.

50. A method of increasing skin rejuvenation of a region of a patient's skin and treating subcutaneous fat associated with said region, comprising:
a. obtaining the system of claim 1,
b. applying either simultaneously or in batch like manner (a) heat to a subcutaneous tissue within said region up to temperature T; (c) radiation in the form of a laser radiation having a wavelength of about 800 nm-1200 nm; or any combination thereof.
